(19) United States
(12) Patent Application Publication
(10) Pub. No.: US 2025/0161403 A1
(43) Pub. Date: May 22, 2025

(54) PEPTIDES AND METHODS OF USE THEREOF IN TREATING OCULAR DISORDERS

(71) Applicant: Biomarck Pharmaceuticals, Ltd., Newtown, PA (US)

(72) Inventors: Amit SHAH, Newtown, PA (US); Graham LUMSDEN, Newtown, PA (US)

(21) Appl. No.: 18/836,300

(22) PCT Filed: Feb. 7, 2023
(86) PCT No.: PCT/US2023/062129

§ 371 Date: Aug. 6, 2024

Related U.S. Application Data
(60) Provisional application No. 63/307,364, filed on Feb. 7, 2022.

(51) Int. Cl. A61K 38/16; A61K 9/00; A61K 38/04; A61K 45/06; A61P 27/02

(52) U.S. Cl. CPC A61K 38/16; A61K 9/0048; A61K 38/04; A61K 45/06; A61P 27/02

(57) ABSTRACT

The present disclosure includes methods of treating an ocular disease or condition at the surface of the eye, including ocular diseases that involve the cornea. More specifically the present disclosure relates to treating an ocular disease or condition such as dry eye syndrome by administering a peptide fragment of the MARCKS protein. Peptide fragments and variants thereof as disclosed in the present disclosure are useful in such methods.

### CROSS REFERENCE TO RELATED APPLICATIONS

This application claims priority from U.S. Provisional Application No. 63/307,364 filed Feb. 7, 2022, which is incorporated by reference herein in its entirety for all purposes.

### TECHNICAL FIELD

The present disclosure relates to peptides or peptide compositions or peptide formulations and methods of their use to treat ocular disorders such as dry eye syndrome. The present disclosure also relates to topical formulations of such peptides.

REFERENCE TO AN ELECTRONIC SEQUENCE LISTING

The contents of the electronic sequence listing (BMRK_008_01WO_SeqList_ST26.xml; Size: 418,796 bytes; and Date of Creation: Feb. 3, 2023) are herein incorporated by reference in its entirety.

### BACKGROUND

Dry eye syndrome (DES), also known as dry eye disease, keratoconjunctivitis sicca (KCS), keratitis sicca, and others, is a disease of the ocular surface. Dry eye syndrome is associated with tear film instability, dysfunction, and/or loss of homeostasis. Dry eye syndrome is a common condition and can be caused by underlying diseases and disorders including autoimmune disorders, or environmental causes. Currently available treatment options for dry eye syndrome are not adequate to treat the disease effectively and/or require repeated administration many times per day. There is a need in the art for improved methods for treating ocular diseases at the surface of the eye such as dry eye syndrome.

### SUMMARY

The disclosure provides a method of treating dry eye syndrome in a subject comprising, administering to said subject a therapeutically effective amount of a composition comprising at least one peptide having an amino acid sequence selected from the group consisting of: (a) an amino acid sequence having from 4 to 24 contiguous amino acids of a reference sequence, GAQFSKTAAKGEAAAERPGEAAVA (SEQ ID NO. 1); (b) an amino acid sequence having the sequence, GAQFSKTAAKGEAAAERPGEAAVA (SEQ ID NO. 1); and (c) an amino acid sequence with at least about 75% identity to the amino acid sequence defined in (a) or (b).

In some aspects, the peptide comprises at least nine contiguous amino acid residues of SEQ ID NO: 1. In some aspects, the peptide comprises an amino acid sequence of SEQ ID NO: 106. In some aspects, the peptide comprises an amino acid sequence of SEQ ID NO: 219. In some aspects, the peptide is myristoylated or acetylated at the N-terminal amino acid. In some aspects, the peptide is acetylated at the N-terminal amino acid, and consists of an amino acid sequence of SEQ ID NO: 106 or 219. In some aspects, the peptide is acetylated at the N-terminal amino acid, and consists of an amino acid sequence of SEQ ID NO: 219. In some aspects, the peptide consists of four contiguous amino acid residues of SEQ ID NO: 1.

In some aspects, the composition comprises a pharmaceutically acceptable carrier. In some aspects, the subject is a mammal. In some aspects, the mammal is selected from the group consisting of humans, canines, equines and felines.

In some aspects, the administering is done by topical administration. In some aspects, the present disclosure provides topical formulations comprising one or more of the peptides disclosed herein. In some aspects, the composition comprises a topical formulation, intra-ocular formulation, an eye implant, an eye drop, eye gel, ointment, microspheres, microemulsion, liposomal formulation or any combination thereof. In some embodiments, the composition is administered by daily topical administrations. In some embodiments, each daily topical administration comprises one, two, three, four, or five topical administrations on each day, for example, approximately one administration every 2, 3, 4, 5, 6, 7, 8, 9, 10, 11, or 12 hours. In some embodiments, on each day that the composition is administered topically, the composition is administered once, twice, or three times.

In some aspects, the method further comprises administration to the subject a second molecule, wherein the second molecule is an antibiotic, an antiviral compound, an antiparasitic compound, an anti-inflammatory compound, an immunomodulatory compound, or any combination thereof.

### BRIEF DESCRIPTION OF THE DRAWINGS

FIG. 1 is a line graph showing corneal opacity scores at baseline, 14 days pre-dose, and at days 1, 3, 7, 14, 21, and 24 after treatment, in each of the following groups: sham control (untreated), PBS control, BIO11006 treated (“MI 006”), and BIO91201 treated (“MI 201”).

FIG. 2 is a line graph showing corneal fluorescein retention scores at baseline, 14 days pre-dose, and at days 1, 3, 7, 14, 21, and 24 after treatment, in each of the following groups: sham control (untreated), PBS control, BIO11006 treated (“MI 006”), and BIO91201 treated (“MI 201”).

FIG. 3 is a line graph showing tear production (mm/20s) at baseline, 14 days pre-dose, and at days 7, 14, 21, and 24 after treatment, in each of the following groups: sham control (untreated), PBS control, BIO11006 treated (“MI 006”), and BIO91201 treated (“MI 201”).

### DETAILED DESCRIPTION

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally the term “about”, as used herein in references to a measurable value such as an amount of weight, time, dose, etc. is meant to encompass values within an acceptable degree of variability in the art. In some embodiments, degree of variability is based on FDA guidelines.

As used herein, “treating” or “treatment” and the like is an approach for obtaining beneficial or desired clinical results. For purposes of this disclosure, beneficial or desired clinical results include, but are not limited to, one or more of the following: alleviation of one or more symptoms of an ocular condition at the surface of the eye such as dry eye syndrome. Symptoms of such an ocular condition include, but are not limited to, pain or discomfort in the eye, dryness in the eye, itchiness in the eye, a burning, stinging, or irritating feeling in the eye or a feeling that a foreign object is in the eye, and sensitivity to light. Accordingly, the terms “treating” or “treatment” and the like include lessening the severity of such symptoms in the eye, including reducing the incidence of, managing, ameliorating, preventing, and/or delaying the development or progression of such symptoms in the eye. Treating or treatment herein can also include improving vision or preventing, stopping, or slowing the progression of vision loss.

The term “effective amount” or “therapeutically effective amount” refers to the amount of an agent that is sufficient to achieve an outcome, for example, to affect beneficial or desired results. The therapeutically effective amount may vary depending upon one or more of: the subject and disease condition being treated, the weight and age of the subject, the severity of the disease condition, the manner of administration and the like.

### MARCKS Protein

MARCKS protein is an actin-binding protein and contributes to cytoskeleton orientation and function, and cell migration. Several exogenous stimuli can provoke degranulation of leukocytes via a pathway that involves activation of protein kinase C and subsequent phosphorylation and dephosphorylation events. MARCKS protein (where MARCKS as used herein means “Myristoylated Alanine-Rich C Kinase Substrate”), is a ubiquitous phosphorylation target of protein kinase C (PKC), and is highly expressed in leukocytes. MARCKS protein is mechanistically involved in a process of exocytotic secretion of mucin by goblet cells that line respiratory airways. MARCKS, a protein of approximately 82 kD, has three evolutionarily-conserved regions, an N-terminus, a phosphorylation site domain (or PSD), and a multiple homology 2 (MH2) domain. MARCKS is myristoylated via an amide bond at the N-terminal amino acid in the MARCKS protein's amino acid sequence at the alpha-amine position of the glycine which resides at the N-terminus (i.e., at position 1) of amino acid sequence via a reaction catalyzed by myristoyl CoA: protein N-myristoyl transferase (NMT). The mechanism appears to involve binding of MARCKS, a myristoylated protein, to membranes of intracellular granules.

The myristoylated N-terminal region of MARCKS appears to be integral to the secretory process because it has been shown to block both mucin secretion and binding of MARCKS to mucin granule membranes in goblet cells. This peptide contains 24 L-amino acids of the MARCKS protein beginning with the N-terminal glycine of the MARCKS protein which is myristoylated via an amide bond and is known as myristoylated alpha-N-terminal sequence (or “MANS”, also interchangeably referred to as the “MARCKS N-terminus”); i.e., Myristoyl-GAQFSKTAAKGEAAAERPGEAAVA (SEQ ID NO: 1). The peptide fragments of the MANS peptide disclosed herein, also preferably are composed of L-amino acids. As MARCKS is an actin-binding protein, it is critical for cytoskeleton orientation and function and cell migration. In some embodiments, the N-terminal MARCKS peptides disclosed herein inhibit directed migration of human neutrophils, fibroblasts, and airway epithelial cells.

Peptides Derived from N-Terminus of MARCKS

The disclosure provides peptides fragments (interchangeably referred to as just “fragments” or just “peptides”) derived from the MARCKS N-terminus. Exemplary MARCKS-related peptide fragments are discussed in U.S. Publication Nos. 2009-0203620 and 2014-0302057, and in International Patent Publication No. WO 2020/257162, the entire contents of each of which are incorporated herein by reference. In some aspects, these peptide fragments play a role in the reducing the rate and/or amount of release of inflammatory mediators granules or vesicles in inflammatory leukocytes.

In some aspects, the peptides disclosed herein are derived from the MARCKS N-terminus, i.e., contiguous peptide fragments derived from within the N-terminal 1-to-24 amino acid sequence of MARCKS. In some aspects, the peptides are N-terminal amides of such fragments, such as N-terminal acetic acid amides of such fragments, and/or as well as C-terminal amides of such fragments, such as C-terminal amides of ammonia. In some aspects, the peptides have from about 4 to about 23 contiguous amino acid residues of the MANS peptide amino acid sequence. In some aspects, the fragments may be N-terminal-myristoylated if they do not begin with the N-terminal glycine at position 1 in SEQ ID NO: 1, or may be N-terminal-acylated with C2 to C12 acyl groups, including N-terminal-acetylated, and/or C-terminal amidated with an NH2 group.

Table 1 contains a list of amino acid sequences in single letter abbreviation format together with a respectively corresponding peptide number and SEQ ID NO. The reference peptide amino acid sequence (MANS peptide) is listed as peptide 1. Amino acid sequences of peptides of the disclosure having an amino acid sequence of from 4 to 23 contiguous amino acids of the reference amino acid sequence are listed as peptides 2 to 231, together with the amino acid sequence of a random N-terminal sequence (RNS) comprising amino acids of the MANS peptide as peptide 232. Amino acid sequences of representative variants of amino acid sequences of peptides of the disclosure as described herein and are also listed as peptides 233 to 245 and 247 to 251. The variant peptides listed are not intended to be a limiting group of peptides, but are presented only to serve as representative examples of variant peptides of the disclosure. Also presented is a representative reverse amino acid sequence (peptide 246) and a representative random amino acid sequence of peptide (peptide 232) of the disclosure.

In some aspects, the peptide comprises an amino acid sequence with at least about 75%, at least about 80%, at least about 85%, at least about 90%, at least about 95%, at least about 96%, at least about 97%, at least about 98%, at least about 99%, or at least about 99.5% identity to any one of the amino acid sequences listed in Table 1. In some aspects, the peptide comprises any one of the amino acid sequences listed in Table 1. In some aspects, the peptides consist of any one of the amino acid sequences listed in Table 1.

TABLE 1
Peptides and Amino Acid Sequences

| Peptide No. | Sequence | Sequence ID No. |
|---|---|---|
| peptide 1 | GAQFSKTAAKGEAAAERPGEAAVA | SEQ ID NO. 1 |
| peptide 2 | GAQFSKTAAKGEAAAERPGEAAV | SEQ ID NO. 2 |
| peptide 3 | AQFSKTAAKGEAAAERPGEAAVA | SEQ ID NO. 3 |
| peptide 4 | GAQFSKTAAKGEAAAERPGEAA | SEQ ID NO. 4 |
| peptide 5 | AQFSKTAAKGEAAAERPGEAAV | SEQ ID NO. 5 |
| peptide 6 | QFSKTAAKGEAAAERPGEAAVA | SEQ ID NO. 6 |
| peptide 7 | GAQFSKTAAKGEAAAERPGEA | SEQ ID NO. 7 |
| peptide 8 | AQFSKTAAKGEAAAERPGEAA | SEQ ID NO. 8 |
| peptide 9 | QFSKTAAKGEAAAERPGEAAV | SEQ ID NO. 9 |
| peptide 10 | FSKTAAKGEAAAERPGEAAVA | SEQ ID NO. 10 |
| peptide 11 | GAQFSKTAAKGEAAAERPGE | SEQ ID NO. 11 |
| peptide 12 | AQFSKTAAKGEAAAERPGEA | SEQ ID NO. 12 |
| peptide 13 | QFSKTAAKGEAAAERPGEAA | SEQ ID NO. 13 |
| peptide 14 | FSKTAAKGEAAAERPGEAAV | SEQ ID NO. 14 |
| peptide 15 | SKTAAKGEAAAERPGEAAVA | SEQ ID NO. 15 |
| peptide 16 | GAQFSKTAAKGEAAAERPG | SEQ ID NO. 16 |
| peptide 17 | AQFSKTAAKGEAAAERPGE | SEQ ID NO. 17 |
| peptide 18 | QFSKTAAKGEAAAERPGEA | SEQ ID NO. 18 |
| peptide 19 | FSKTAAKGEAAAERPGEAA | SEQ ID NO. 19 |
| peptide 20 | SKTAAKGEAAAERPGEAAV | SEQ ID NO. 20 |
| peptide 21 | KTAAKGEAAAERPGEAAVA | SEQ ID NO. 21 |
| peptide 22 | GAQFSKTAAKGEAAAERP | SEQ ID NO. 22 |
| peptide 23 | AQFSKTAAKGEAAAERPG | SEQ ID NO. 23 |
| peptide 24 | QFSKTAAKGEAAAERPGE | SEQ ID NO. 24 |
| peptide 25 | FSKTAAKGEAAAERPGEA | SEQ ID NO. 25 |
| peptide 26 | SKTAAKGEAAAERPGEAA | SEQ ID NO. 26 |
| peptide 27 | KTAAKGEAAAERPGEAAV | SEQ ID NO. 27 |
| peptide 28 | TAAKGEAAAERPGEAAVA | SEQ ID NO. 28 |
| peptide 29 | GAQFSKTAAKGEAAAER | SEQ ID NO. 29 |
| peptide 30 | AQFSKTAAKGEAAAERP | SEQ ID NO. 30 |
| peptide 31 | QFSKTAAKGEAAAERPG | SEQ ID NO. 31 |
| peptide 32 | FSKTAAKGEAAAERPGE | SEQ ID NO. 32 |
| peptide 33 | SKTAAKGEAAAERPGEA | SEQ ID NO. 33 |
| peptide 34 | KTAAKGEAAAERPGEAA | SEQ ID NO. 34 |
| peptide 35 | TAAKGEAAAERPGEAAV | SEQ ID NO. 35 |
| peptide 36 | AAKGEAAAERPGEAAVA | SEQ ID NO. 36 |
| peptide 37 | GAQFSKTAAKGEAAAE | SEQ ID NO. 37 |
| peptide 38 | AQFSKTAAKGEAAAER | SEQ ID NO. 38 |
| peptide 39 | QFSKTAAKGEAAAERP | SEQ ID NO. 39 |
| peptide 40 | FSKTAAKGEAAAERPG | SEQ ID NO. 40 |
| peptide 41 | SKTAAKGEAAAERPGE | SEQ ID NO. 41 |
| peptide 42 | KTAAKGEAAAERPGEA | SEQ ID NO. 42 |
| peptide 43 | TAAKGEAAAERPGEAA | SEQ ID NO. 43 |
| peptide 44 | AAKGEAAAERPGEAAV | SEQ ID NO. 44 |
| peptide 45 | AKGEAAAERPGEAAVA | SEQ ID NO. 45 |
| peptide 46 | GAQFSKTAAKGEAAA | SEQ ID NO. 46 |
| peptide 47 | AQFSKTAAKGEAAAE | SEQ ID NO. 47 |
| peptide 48 | QFSKTAAKGEAAAER | SEQ ID NO. 48 |
| peptide 49 | FSKTAAKGEAAAERP | SEQ ID NO. 49 |
| peptide 50 | SKTAAKGEAAAERPG | SEQ ID NO. 50 |
| peptide 51 | KTAAKGEAAAERPGE | SEQ ID NO. 51 |
| peptide 52 | TAAKGEAAAERPGEA | SEQ ID NO. 52 |
| peptide 53 | AAKGEAAAERPGEAA | SEQ ID NO. 53 |
| peptide 54 | AKGEAAAERPGEAAV | SEQ ID NO. 54 |
| peptide 55 | KGEAAAERPGEAAVA | SEQ ID NO. 55 |
| peptide 56 | GAQFSKTAAKGEAA | SEQ ID NO. 56 |
| peptide 57 | AQFSKTAAKGEAAA | SEQ ID NO. 57 |
| peptide 58 | QFSKTAAKGEAAAE | SEQ ID NO. 58 |
| peptide 59 | FSKTAAKGEAAAER | SEQ ID NO. 59 |
| peptide 60 | SKTAAKGEAAAERP | SEQ ID NO. 60 |
| peptide 61 | KTAAKGEAAAERPG | SEQ ID NO. 61 |
| peptide 62 | TAAKGEAAAERPGE | SEQ ID NO. 62 |
| peptide 63 | AAKGEAAAERPGEA | SEQ ID NO. 63 |
| peptide 64 | AKGEAAAERPGEAA | SEQ ID NO. 64 |
| peptide 65 | KGEAAAERPGEAAV | SEQ ID NO. 65 |
| peptide 66 | GEAAAERPGEAAVA | SEQ ID NO. 66 |
| peptide 67 | GAQFSKTAAKGEA | SEQ ID NO. 67 |
| peptide 68 | AQFSKTAAKGEAA | SEQ ID NO. 68 |
| peptide 69 | QFSKTAAKGEAAA | SEQ ID NO. 69 |
| peptide 70 | FSKTAAKGEAAAE | SEQ ID NO. 70 |
| peptide 71 | SKTAAKGEAAAER | SEQ ID NO. 71 |
| peptide 72 | KTAAKGEAAAERP | SEQ ID NO. 72 |
| peptide 73 | TAAKGEAAAERPG | SEQ ID NO. 73 |
| peptide 74 | AAKGEAAAERPGE | SEQ ID NO. 74 |
| peptide 75 | AKGEAAAERPGEA | SEQ ID NO. 75 |
| peptide 76 | KGEAAAERPGEAA | SEQ ID NO. 76 |
| peptide 77 | GEAAAERPGEAAV | SEQ ID NO. 77 |
| peptide 78 | EAAAERPGEAAVA | SEQ ID NO. 78 |
| peptide 79 | GAQFSKTAAKGE | SEQ ID NO. 79 |
| peptide 80 | AQFSKTAAKGEA | SEQ ID NO. 80 |
| peptide 81 | QFSKTAAKGEAA | SEQ ID NO. 81 |
| peptide 82 | FSKTAAKGEAAA | SEQ ID NO. 82 |
| peptide 83 | SKTAAKGEAAAE | SEQ ID NO. 83 |
| peptide 84 | KTAAKGEAAAER | SEQ ID NO. 84 |
| peptide 85 | TAAKGEAAAERP | SEQ ID NO. 85 |
| peptide 86 | AAKGEAAAERPG | SEQ ID NO. 86 |
| peptide 87 | AKGEAAAERPGE | SEQ ID NO. 87 |
| peptide 88 | KGEAAAERPGEA | SEQ ID NO. 88 |
| peptide 89 | GEAAAERPGEAA | SEQ ID NO. 89 |
| peptide 90 | EAAAERPGEAAV | SEQ ID NO. 90 |
| peptide 91 | AAAERPGEAAVA | SEQ ID NO. 91 |
| peptide 92 | GAQFSKTAAKG | SEQ ID NO. 92 |
| peptide 93 | AQFSKTAAKGE | SEQ ID NO. 93 |
| peptide 94 | QFSKTAAKGEA | SEQ ID NO. 94 |
| peptide 95 | FSKTAAKGEAA | SEQ ID NO. 95 |
| peptide 96 | SKTAAKGEAAA | SEQ ID NO. 96 |
| peptide 97 | KTAAKGEAAAE | SEQ ID NO. 97 |
| peptide 98 | TAAKGEAAAER | SEQ ID NO. 98 |
| peptide 99 | AAKGEAAAERP | SEQ ID NO. 99 |
| peptide 100 | AKGEAAAERPG | SEQ ID NO. 100 |
| peptide 101 | KGEAAAERPGE | SEQ ID NO. 101 |
| peptide 102 | GEAAAERPGEA | SEQ ID NO. 102 |
| peptide 103 | EAAAERPGEAA | SEQ ID NO. 103 |
| peptide 104 | AAAERPGEAAV | SEQ ID NO. 104 |
| peptide 105 | AAERPGEAAVA | SEQ ID NO. 105 |
| peptide 106 | GAQFSKTAAK | SEQ ID NO. 106 |
| peptide 107 | AQFSKTAAKG | SEQ ID NO. 107 |
| peptide 108 | QFSKTAAKGE | SEQ ID NO. 108 |
| peptide 109 | FSKTAAKGEA | SEQ ID NO. 109 |
| peptide 110 | SKTAAKGEAA | SEQ ID NO. 110 |
| peptide 111 | KTAAKGEAAA | SEQ ID NO. 111 |
| peptide 112 | TAAKGEAAAE | SEQ ID NO. 112 |
| peptide 113 | AAKGEAAAER | SEQ ID NO. 113 |
| peptide 114 | AKGEAAAERP | SEQ ID NO. 114 |
| peptide 115 | KGEAAAERPG | SEQ ID NO. 115 |
| peptide 116 | GEAAAERPGE | SEQ ID NO. 116 |
| peptide 117 | EAAAERPGEA | SEQ ID NO. 117 |
| peptide 118 | AAAERPGEAA | SEQ ID NO. 118 |
| peptide 119 | AAERPGEAAV | SEQ ID NO. 119 |
| peptide 120 | AERPGEAAVA | SEQ ID NO. 120 |
| peptide 121 | GAQFSKTAA | SEQ ID NO. 121 |
| peptide 122 | AQFSKTAAK | SEQ ID NO. 122 |
| peptide 123 | QFSKTAAKG | SEQ ID NO. 123 |
| peptide 124 | FSKTAAKGE | SEQ ID NO. 124 |
| peptide 125 | SKTAAKGEA | SEQ ID NO. 125 |
| peptide 126 | KTAAKGEAA | SEQ ID NO. 126 |
| peptide 127 | TAAKGEAAA | SEQ ID NO. 127 |
| peptide 128 | AAKGEAAAE | SEQ ID NO. 128 |
| peptide 129 | AKGEAAAER | SEQ ID NO. 129 |
| peptide 130 | KGEAAAERP | SEQ ID NO. 130 |
| peptide 131 | GEAAAERPG | SEQ ID NO. 131 |
| peptide 132 | EAAAERPGE | SEQ ID NO. 132 |
| peptide 133 | AAAERPGEA | SEQ ID NO. 133 |
| peptide 134 | AAERPGEAA | SEQ ID NO. 134 |
| peptide 135 | AERPGEAAV | SEQ ID NO. 135 |
| peptide 136 | ERPGEAAVA | SEQ ID NO. 136 |
| peptide 137 | GAQFSKTA | SEQ ID NO. 137 |
| peptide 138 | AQFSKTAA | SEQ ID NO. 138 |
| peptide 139 | QFSKTAAK | SEQ ID NO. 139 |
| peptide 140 | FSKTAAKG | SEQ ID NO. 140 |
| peptide 141 | SKTAAKGE | SEQ ID NO. 141 |
| peptide 142 | KTAAKGEA | SEQ ID NO. 142 |
| peptide 143 | TAAKGEAA | SEQ ID NO. 143 |
| peptide 144 | AAKGEAAA | SEQ ID NO. 144 |
| peptide 145 | AKGEAAAE | SEQ ID NO. 145 |
| peptide 146 | KGEAAAER | SEQ ID NO. 146 |
| peptide 147 | GEAAAERP | SEQ ID NO. 147 |
| peptide 148 | EAAAERPG | SEQ ID NO. 148 |
| peptide 149 | AAAERPGE | SEQ ID NO. 149 |
| peptide 150 | AAERPGEA | SEQ ID NO. 150 |
| peptide 151 | AERPGEAA | SEQ ID NO. 151 |
| peptide 152 | ERPGEAAV | SEQ ID NO. 152 |
| peptide 153 | RPGEAAVA | SEQ ID NO. 153 |
| peptide 154 | GAQFSKT | SEQ ID NO. 154 |
| peptide 155 | AQFSKTA | SEQ ID NO. 155 |
| peptide 156 | QFSKTAA | SEQ ID NO. 156 |
| peptide 157 | FSKTAAK | SEQ ID NO. 157 |
| peptide 158 | SKTAAKG | SEQ ID NO. 158 |
| peptide 159 | KTAAKGE | SEQ ID NO. 159 |
| peptide 160 | TAAKGEA | SEQ ID NO. 160 |
| peptide 161 | AAKGEAA | SEQ ID NO. 161 |
| peptide 162 | AKGEAAA | SEQ ID NO. 162 |
| peptide 163 | KGEAAAE | SEQ ID NO. 163 |
| peptide 164 | GEAAAER | SEQ ID NO. 164 |
| peptide 165 | EAAAERP | SEQ ID NO. 165 |
| peptide 166 | AAAERPG | SEQ ID NO. 166 |
| peptide 167 | AAERPGE | SEQ ID NO. 167 |
| peptide 168 | AERPGEA | SEQ ID NO. 168 |
| peptide 169 | ERPGEAA | SEQ ID NO. 169 |
| peptide 170 | RPGEAAV | SEQ ID NO. 170 |
| peptide 171 | PGEAAVA | SEQ ID NO. 171 |
| peptide 172 | GAQFSK | SEQ ID NO. 172 |
| peptide 173 | AQFSKT | SEQ ID NO. 173 |
| peptide 174 | QFSKTA | SEQ ID NO. 174 |
| peptide 175 | FSKTAA | SEQ ID NO. 175 |
| peptide 176 | SKTAAK | SEQ ID NO. 176 |
| peptide 177 | KTAAKG | SEQ ID NO. 177 |
| peptide 178 | TAAKGE | SEQ ID NO. 178 |
| peptide 179 | AAKGEA | SEQ ID NO. 179 |
| peptide 180 | AKGEAA | SEQ ID NO. 180 |
| peptide 181 | KGEAAA | SEQ ID NO. 181 |
| peptide 182 | GEAAAE | SEQ ID NO. 182 |
| peptide 183 | EAAAER | SEQ ID NO. 183 |
| peptide 184 | AAAERP | SEQ ID NO. 184 |
| peptide 185 | AAERPG | SEQ ID NO. 185 |
| peptide 186 | AERPGE | SEQ ID NO. 186 |
| peptide 187 | ERPGEA | SEQ ID NO. 187 |
| peptide 188 | RPGEAA | SEQ ID NO. 188 |
| peptide 189 | PGEAAV | SEQ ID NO. 189 |
| peptide 190 | GEAAVA | SEQ ID NO. 190 |
| peptide 191 | GAQFS | SEQ ID NO. 191 |
| peptide 192 | AQFSK | SEQ ID NO. 192 |
| peptide 193 | QFSKT | SEQ ID NO. 193 |
| peptide 194 | FSKTA | SEQ ID NO. 194 |
| peptide 195 | SKTAA | SEQ ID NO. 195 |
| peptide 196 | KTAAK | SEQ ID NO. 196 |
| peptide 197 | TAAKG | SEQ ID NO. 197 |
| peptide 198 | AAKGE | SEQ ID NO. 198 |
| peptide 199 | AKGEA | SEQ ID NO. 199 |
| peptide 200 | KGEAA | SEQ ID NO. 200 |
| peptide 201 | GEAAA | SEQ ID NO. 201 |
| peptide 202 | EAAAE | SEQ ID NO. 202 |
| peptide 203 | AAAER | SEQ ID NO. 203 |
| peptide 204 | AAERP | SEQ ID NO. 204 |
| peptide 205 | AERPG | SEQ ID NO. 205 |
| peptide 206 | ERPGE | SEQ ID NO. 206 |
| peptide 207 | RPGEA | SEQ ID NO. 207 |
| peptide 208 | PGEAA | SEQ ID NO. 208 |
| peptide 209 | GEAAV | SEQ ID NO. 209 |
| peptide 210 | EAAVA | SEQ ID NO. 210 |
| peptide 211 | GAQF | SEQ ID NO. 211 |
| peptide 212 | AQFS | SEQ ID NO. 212 |
| peptide 213 | QFSK | SEQ ID NO. 213 |
| peptide 214 | FSKT | SEQ ID NO. 214 |
| peptide 215 | SKTA | SEQ ID NO. 215 |
| peptide 216 | KTAA | SEQ ID NO. 216 |
| peptide 217 | TAAK | SEQ ID NO. 217 |
| peptide 218 | AAKG | SEQ ID NO. 218 |
| peptide 219 | AKGE | SEQ ID NO. 219 |
| peptide 220 | KGEA | SEQ ID NO. 220 |
| peptide 221 | GEAA | SEQ ID NO. 221 |
| peptide 222 | EAAA | SEQ ID NO. 222 |
| peptide 223 | AAAE | SEQ ID NO. 223 |
| peptide 224 | AAER | SEQ ID NO. 224 |
| peptide 225 | AERP | SEQ ID NO. 225 |
| peptide 226 | ERPG | SEQ ID NO. 226 |
| peptide 227 | RPGE | SEQ ID NO. 227 |
| peptide 228 | PGEA | SEQ ID NO. 228 |
| peptide 229 | GEAA | SEQ ID NO. 229 |
| peptide 230 | EAAV | SEQ ID NO. 230 |
| peptide 231 | AAVA | SEQ ID NO. 231 |
| peptide 232 | GTAPAAEGAGAEVKRASAEAKQAF | SEQ ID NO. 232 |
| peptide 233 | GKQFSKTAAKGE | SEQ ID NO. 233 |
| peptide 234 | GAQFSKTKAKGE | SEQ ID NO. 234 |
| peptide 235 | GKQFSKTKAKGE | SEQ ID NO. 235 |
| peptide 236 | GAQASKTAAK | SEQ ID NO. 236 |
| peptide 237 | GAQASKTAAKGE | SEQ ID NO. 237 |
| peptide 238 | GAEFSKTAAKGE | SEQ ID NO. 238 |
| peptide 239 | GAQFSKTAAAGE | SEQ ID NO. 239 |
| peptide 240 | GAQFSKTAAKAE | SEQ ID NO. 240 |
| peptide 241 | GAQFSKTAAKGA | SEQ ID NO. 241 |
| peptide 242 | AAQFSKTAAK | SEQ ID NO. 242 |
| peptide 243 | GAAFSKTAAK | SEQ ID NO. 243 |
| peptide 244 | GAQFAKTAAK | SEQ ID NO. 244 |
| peptide 245 | GAQFSATAAK | SEQ ID NO. 245 |
| peptide 246 | KAATKSFQAG | SEQ ID NO. 246 |
| peptide 247 | GAQFSKAAAK | SEQ ID NO. 247 |
| peptide 248 | GAQFSKTAAA | SEQ ID NO. 248 |
| peptide 249 | GAQFSATAAA | SEQ ID NO. 249 |
| peptide 250 | GAQASKTA | SEQ ID NO. 250 |
| peptide 251 | AAGE | SEQ ID NO. 251 |
| peptide 252 | GKASQFAKTA | SEQ ID NO. 252 |

In some aspects, the peptide is any one of the peptides listed in Table 1A.

TABLE 1A

| Peptide Name | Seq ID NO. |
|---|---|
| MANS | 1 |
| Ac-MANS | 1 |
| BIO-11211 (Ac-----------NH2) | 79 |
| BIO-11000 (Ma-----------OH) | 106 |
| BIO-11002 (Ma-----------NH2) | 106 |
| BIO-11005 (H------------NH2 | 106 |
| BIO-11006 (Ac---------OH) | 106 |
| BIO-11007 (cyclic) | 106 cyclic |
| BIO-11018 (pegylated) | 106 pegylated |
| BIO-11026 (Ac-----------NH2) | 106 |
| BIO-10901 (Ac-----------OH) | 121 |
| BIO-10803 (Ac--------OH) | 137 |
| BIO-91200 (Ma-AKGE-OH) | 219 |
| BIO-91201 (Ac-AKGE-OH) | 219 |
| BIO-91202 (Ac-AKGE-NH2) | 219 |

Ma = Myristoyl;
Ac = Acetyl

The disclosure provides peptides having amino acid sequences comprising less than 24 amino acids with amino acid sequences related to the amino acid sequence of MANS peptide. The peptides of the current disclosure consist of amino acid sequences containing less than 24 amino acids, and may consist of from 4 to 14, from 10 to 12, from 9 to 14, from 9 to 13, from 10 to 13, from 10 to 14, at least 4, at least 9, at least 10, or the like amino acids. The peptides are typically straight chains, but may be cyclic peptides as well. Cyclic peptides are peptides that contain a circular or cyclic ring structure. The circular ring structure can be formed, for example, through connection between the amino and carboxyl ends of the peptide, or between the carboxyl or amino end and a side chain, or between a peptide backbone and the carboxyl or amino end or a side chain, or between two positions on the peptide backbone, or between two side chains. The connections may be formed via an amide bond, or other chemically stable bonds. In some embodiments, the peptide is a head-to-tail cyclic peptide. In some embodiments, the peptides are pegylated (PEGylated). PEGylating is the process of covalently attaching polyethylene glycol (PEG) chains to peptides. In some embodiments, PEGylating enhances solubility and/or half life of peptides, and/or reduces immunogenicity. Thus, in some embodiments, peptide PEGylation therapeutic efficacy and/or tolerability of peptide drugs. In some embodiments, the peptides are synthetic peptides. In some embodiments, the peptides are isolated peptides.

In some aspects, the peptide has an amino acid sequence selected from the group consisting of (a) an amino acid sequence having from 4 to 23 contiguous amino acids of the reference sequence, peptide 1; (b) a sequence with at least about 75%, at least about 80% identity, at least about 85%, at least about 90%, at least about 91%, at least about 92%, at least about 93%, at least about 94%, or at least about 95% identity to the amino acid sequence defined in (a); or (c) a variant of the amino acid sequence defined in (a), which variant is selected from the group consisting of a substitution variant, a deletion variant, an addition variant, and combinations thereof.

In other embodiments, the amino acid sequence of the peptide does not begin at the N-terminal amino acid of the reference sequence, peptide 1, (SEQ ID NO: 1) but rather begins at the amino acid at position 2 through the amino acid at position 21 of the reference sequence peptide 1. For example, the peptides may have an amino acid sequence selected from the group consisting of (a) an amino acid sequence having from 4 to 23 contiguous amino acids of the reference sequence peptide 1, wherein the amino acid sequence begins at any amino acid between position 2 through position 21 of the reference sequence. These peptides may be between 4 and 23 contiguous amino acids long and may represent peptides in the middle of the reference sequence, peptide 1; (b) a sequence with at least about 75%, at least 80% about identity, at least about 85%, at least about 90%, at least about 91%, at least about 92%, at least about 93%, at least about 94%, or at least about 95% identity to the amino acid sequence defined in (a); or (c) a variant of the amino acid sequence defined in (a). These peptides may contain no covalently bound chemical moiety or a chemical moiety on the N-terminal amino acid which is not the N-terminal glycine from or equivalent to the N-terminal glycine of the amino acid sequence SEQ ID NO: 1. Preferably, the chemical moiety is an acyl group, such as an acetyl group or a myristoyl group, in the form of an amide bond, or an alkyl group.

In yet other embodiments, the amino acid sequence of the peptide includes the contiguous residues A, K, G, and E as in peptide 219 of the reference sequence peptide 1. For example, the peptides may have an amino acid sequence selected from the group consisting of (a) an amino acid sequence having from 4 to 23 contiguous amino acids of the reference sequence peptide 1, wherein the amino acid sequence of the peptide includes the contiguous residues A, K, G, and E as in peptide 219 of the reference peptide 1 (e.g., peptide 219, peptide 45, peptide 79, peptide 67, peptide 80, etc.); (b) a sequence with at least about 75%, at least about 80% identity, at least about 85%, at least about 90%, at least about 91%, at least about 92%, at least about 93%, at least about 94%, or at least about 95% identity to the amino acid sequence defined in (a); or (c) a variant of the amino acid sequence defined in (a).

In further embodiments, the amino acid sequence of the peptide begins from the N-terminal amino acid of the reference sequence peptide 1 and includes the contiguous residues A, K, G, and E as in peptide 219 of the reference sequence peptide 1, while in other embodiments the amino acid sequence of the peptide ends at the C-terminal amino acid of the reference sequence peptide 1 and includes the contiguous residues A, K, G, and E as in peptide 219 of the reference sequence peptide 1. In some embodiments, the amino acid sequence of the peptide consists of SEQ ID NO: 219.

In exemplary aspects, the peptide is acetylated at the N-terminal amino acid. In exemplary aspects, the peptide comprises or consists of the amino acid sequence of SEQ ID NO: 106, and is acetylated at the N-terminal amino acid. In some aspects, the peptide comprises or consists of the amino acid sequence of SEQ ID NO: 219, and is acetylated at the N-terminal amino acid.

In some embodiments, the present disclosure provides composition comprising the peptides provided herein and salts thereof. For example, in some embodiments, the disclosure encompasses the peptides provided herein and pharmaceutically acceptable salts thereof. Pharmaceutically acceptable salts of the peptides of this disclosure include, for example, peptides modified by making acid or base salts thereof. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

### Pharmaceutical Compositions

In some aspects, any one of the peptides disclosed herein is contained in a pharmaceutical composition which is useful to prevent, treat, and/or block progression of dry eye syndrome.

The disclosure also encompasses a composition comprising a peptide as described in the paragraphs above and described herein and an excipient. The disclosure also encompasses a pharmaceutical composition comprising a peptide as described in the paragraphs above and described herein and a pharmaceutically acceptable carrier. The pharmaceutical composition can further preferably be sterile, sterilizable or sterilized. These peptides can be contained in a kit with reagents useful for administration.

In one aspect, the disclosure relates to a method of administering a pharmaceutical composition. The pharmaceutical composition comprises a therapeutically effective amount of a known compound and a pharmaceutically acceptable carrier. Pharmaceutically acceptable carriers are preferably liquid dosage forms. Liquid preparations may be used and may be prepared in the form of solutions or suspensions, e.g., solutions containing an active ingredient, and a mixture of water, glycerol, and propylene glycol. If desired, such liquid preparations may include one or more of following: thickening agents such as carboxymethylcellulose also may be used as well as other acceptable carriers, the selection of which is known in the art.

In certain embodiments, the drug product is present in a solid pharmaceutical composition. A solid composition of matter according to the present disclosure may be formed and may be mixed with and/or diluted by an excipient. The solid composition of matter also may be enclosed within a carrier, which may be, for example, in the form of a capsule, sachet, tablet, paper, or other container. When the excipient serves as a diluent, it may be a solid, semi-solid, a gel, or liquid material that acts as a vehicle, carrier, or medium for the composition of matter. For ophthalmic administration, the pharmaceutical formulation with any one of the peptides disclosed herein can be prepared in the form of an eye drop, eye gel, ointment, ointment, implant, microspheres, or liposomal formulation, or microemulsion.

Various suitable excipients will be understood by those skilled in the art and may be found in the National Formulary, 19:2404-2406 (2000), the disclosure of pages 2404 to 2406 being incorporated herein in their entirety. Examples of suitable excipients include, but are not limited to, starches, gum arabic, calcium silicate, microcrystalline cellulose, methacrylates, shellac, polyvinylpyrrolidone, cellulose, water, and methylcellulose. The drug product formulations additionally can include lubricating agents such as, for example, talc, magnesium stearate and mineral oil; wetting agents; emulsifying and suspending agents; preserving agents such as methyl- and propyl hydroxybenzoates. Polyols, buffers, and inert fillers also may be used. Examples of polyols include, but are not limited to, mannitol, sorbitol, xylitol, sucrose, maltose, glucose, lactose, dextrose, and the like. Suitable buffers include, but are not limited to, phosphate, citrate, tartrate, succinate, and the like. Other inert fillers that may be used include those that are known in the art and are useful in the manufacture of various dosage forms. If desired, the solid formulations may include other components such as bulking agents and/or granulating agents, and the like. The drug products of the disclosure may be formulated so as to provide quick, sustained, or delayed release of the active ingredient after administration to the patient by employing procedures well known in the art.

In the event that the above pharmaceuticals are to be used for parenteral or intra-ocular administration, such a formulation may comprise sterile aqueous injection solutions, non-aqueous injection solutions, or both, comprising the composition of matter of the present disclosure. When aqueous injection solutions are prepared, the composition of matter may be present as a water soluble pharmaceutically acceptable salt. Parenteral or intra-ocular preparations may contain anti-oxidants, buffers, bacteriostats, and solutes which render the formulation isotonic with the blood of the intended recipient. Aqueous and non-aqueous sterile suspensions may comprise suspending agents and thickening agents. The formulations may be presented in unit-dose or multi-dose containers, for example sealed ampules and vials. Extemporaneous injection solutions and suspensions may be prepared from sterile powders, granules and tablets of the kind previously described. The parenteral or intra-ocular formulation can also be as liposomal composition.

The composition of matter also may be formulated such that it may be suitable for topical administration (e.g., ophthalmic drop or gel, or cream). These formulations may contain various excipients known to those skilled in the art. Suitable excipients may include, but are not limited to, cetyl esters wax, cetyl alcohol, white wax, glyceryl monostearate, propylene glycol, monostearate, methyl stearate, benzyl alcohol, sodium lauryl sulfate, glycerin, mineral oil, water, carbomer, ethyl alcohol, acrylate adhesives, polyisobutylene adhesives, and silicone adhesives.

Methods of Treating Dry Eye Syndrome and/or Other Corneal Conditions

Dry eye syndrome, dry eye disease, and keratoconjucintivitis sicca are used interchangeably herein. Dry eye syndrome can be any syndrome associated with dysfunction and/or instability of tear film, including reduced tear secretion and increased tear evaporation. Indications encompassed by the term dry eye syndrome an the like include age-related dry eye, Stevens-Johnson syndrome, Sjogren's syndrome, ocular cicatrical pemphigoid, corneal injury, ocular surface infection, Riley-Day syndrome, congenital alacrima, nutritional disorders or deficiencies (including vitamin deficiencies), pharmacologic side effects, glandular and tissue destruction, autoimmune and other immunodeficient disorders, and inability to blink in comatose patients. Also included are dry eye symptoms caused by environmental exposure to airborne particulates, smoke, smog, and excessively dry air; as well as contact lens intolerance and eye stress caused by computer work or computer gaming.

Allergic conjunctivitis (seasonal and chronic), blepharitis and Meibomian gland dysfunction are examples of conditions that affect the quality and stability of the tear film, which results in dry eye signs and symptoms. Laser assisted vision correction procedures such as photorefractive keratectomy (PRK), laser-assisted sub-epithelial keratectomy (LASEK) and laser-assisted in situ keratomileusis (LASIK) can also negatively influence tear film functionality and cause dry eye disease.

The disclosure provides methods of treating dry eye syndrome in a subject by administering to the subject any one of the peptides disclosed herein. In some aspects, the method comprising administering to the subject any one of the peptides listed in Table 1 or Table 1A. In some embodiments, the peptide is present in a topical formulation for administration to the surface of the eye. The disclosure further provides methods of treating dry eye syndrome in a subject by administering to the subject a composition comprising any one of the peptides disclosed herein. In embodiments, the peptide comprises or consists of the sequence of SEQ ID NO: 106 or 219.

In some aspects, the subject is a mammal, such as humans, canines, equines and felines.

The method of administration of the peptides and compositions disclosed herein may be by topical administration. The ophthalmic administration generally includes an eye drop, eye gel, ointment, ointment, implant, microspheres, or liposomal formulation. In some embodiments, the method of administration of the peptides and compositions disclosed herein is by IVT injection. In some embodiments, the method of administration of the peptides and compositions disclosed herein is by a combination of IVT injection and topical administration. For example, in some embodiments, the compositions are administered by IVT injection followed by topical administration; or by topical administration followed by IVT injection. Topical administration may be, for example, once daily, twice daily, three times daily, four times daily, or more.

Additionally, the administration to the subject can further include the administration of a second molecule selected from the group consisting of an antibiotic, an antiviral compound, an antiparasitic compound, an anti-inflammatory compound, and an immunomodulator. As used herein, an immunomodulator or immunomodulatory compound is an agent that can affect the functioning of the immune system. In some aspects, the immunomodulatory compound helps normalize or regulate the immune system. Non limiting examples of immunomodulators include azathioprine, methotrexate, cyclosporine, tacrolimus, sirolimus, and everolimus.

In some aspects, the peptide is administered at a concentration from about 1 M to about 10 mM, such as, for example, about 10 μM, about 20 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 950 μM, about 1 mM, about 2 mM, about 3 mM, about 4 mM, about 5 mM, about 6 mM, about 7 mM, about 8 mM, about 9 mM, or about 10 mM, including all subranges and values that lie therebetween.

In some aspects, the peptide is administered in an amount of about lug to about 5 mg, such as for example, about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1 mg, about 2 mg, about 3 mg, about 4 mg, or about 5 mg, including all subranges and values that lie therebetween.

In some embodiments, the peptide may be administered in a volume of about 0.01 mL to about 1 mL, such as for example, about 0.01 mL, about 0.05 mL, about 0.1 mL, about 0.5 mL, about 0.75 mL, or about 1 mL, including all subranges and values that lie therebetween.

The disclosure also provides a topical composition or topical formulation comprising at least one peptide having an amino acid sequence selected from the group consisting of: (a) an amino acid sequence having from 4 to 24 contiguous amino acids of a reference sequence, GAQFSKTAAKGEAAAERPGEAAVA (SEQ ID NO. 1); (b) an amino acid sequence having the sequence, GAQFSKTAAKGEAAAERPGEAAVA (SEQ ID NO. 1); and (c) an amino acid sequence with at least about 75% identity to the amino acid sequence defined in (a) or (b), for use in a method of treating dry eye syndrome in a subject, the method comprising administering the composition to the subject. In embodiments, the peptide has an amino acid sequence according to SEQ ID NO: 106 or SEQ ID NO: 219. In embodiments, the peptide is BIO-91201 (Ac-AKGE-OH; SEQ ID NO: 219). In embodiments, the topical composition or topical formulation is suitable for administration to the surface of the eye.

Having now described the disclosure, the same will be illustrated with reference to certain examples, which are included herein for illustration purposes only, and which are not intended to be limiting of the disclosure.

### EXAMPLES

### Example 1. Topical Peptide Inhibitors of MARCKS Reduce Signs of Dry Eye in the Alacrimal Rat Model

Dry eye was induced in the left eye of Sprague-Dawley rats by removal of the extraorbital lacrimal gland. One group of rats had a skin incision, but no lacrimal gland removal (sham group). Topical administration of test compounds was initiated 2 weeks after surgery, given 4 times a day and continued for 24 days. Sham rats (n=6) were untreated. Alacrimal rats (n=6 each) were dosed bilaterally with 10 μL of phosphate buffered saline (PBS), or 100 μM of either of two MARCKS inhibitors: BIO-11006 [high molecular weight] or BIO-91201 [lower molecular weight]. The efficacy of these MARCKS inhibitor peptides was assessed via clinical examination using biomicroscopy and phenol red test (PRT) tear volume with the observers masked to the treatments. Examinations were at baseline, 2 weeks after surgery, and on days 1, 3, 7, 14, 21, and 24 after initiating dosing. Clinical scores for corneal opacity, vascularization, and corneal fluorescein (FL) retention were recorded at each examination. The experimental design of the study is provided in the table below.

| | | | Test Article | | Time of Euthanasia (4 |
|---|---|---|---|---|---|
| | # of | Surgical | (10 μL | Experimental | weeks after initiating |
| Group | Animals | procedure | topical q6hr) | Endpoints | treatment) |
| 1 | 6 | Sham OS | None | Ocular exams, | Serum collected |
| | | (incision but | | fluorescein dye, | Eyes removed and |
| | | no lacrimal | | and tear testing | fixed in 10% NBF |
| | | gland removal) | | (PRT): Baseline, | |
| 2 | 6 | Extraorbital | Saline OU | prior to initiating | |
| | | lacrimal gland | | treatment, then 1, | |
| | | removal OS | | 3, 7, and then | |
| 2 | 6 | Extraorbital | BIO-11006 100 | weekly for 4 | |
| | | lacrimal gland | uM OU | weeks | |
| | | removal OS | | | |
| 4 | 6 | Extraorbital | BIO-91201 100 | | |
| | | lacrimal gland | uM OU | | |
| | | removal OS | | | |

Clinical signs of dry eye were observed 2 weeks after surgery in groups with lacrimal gland removal, but not in rats in the sham group. No clinical abnormalities were observed in the right eyes (non-surgical) of the rats throughout the study. Left eyes of rats in the sham group had significantly lower clinical scores and higher PRT values than the PBS-dosed group at each examination day after surgery.

Corneal scores were assessed as in the table below.

| 0 | None |
|---|---|
| 1 | minimal superficial opacity |
| 2 | mild deep (stromal) opacity with pupil |
| | margin and iris vessels visible |
| 3 | moderate stromal opacity with only pupil |
| | margin visible |
| 4 | intense stromal opacity with anterior |
| | chamber visible |
| 5 | maximal corneal opacity with total |
| | obscuration of the anterior chamber. |

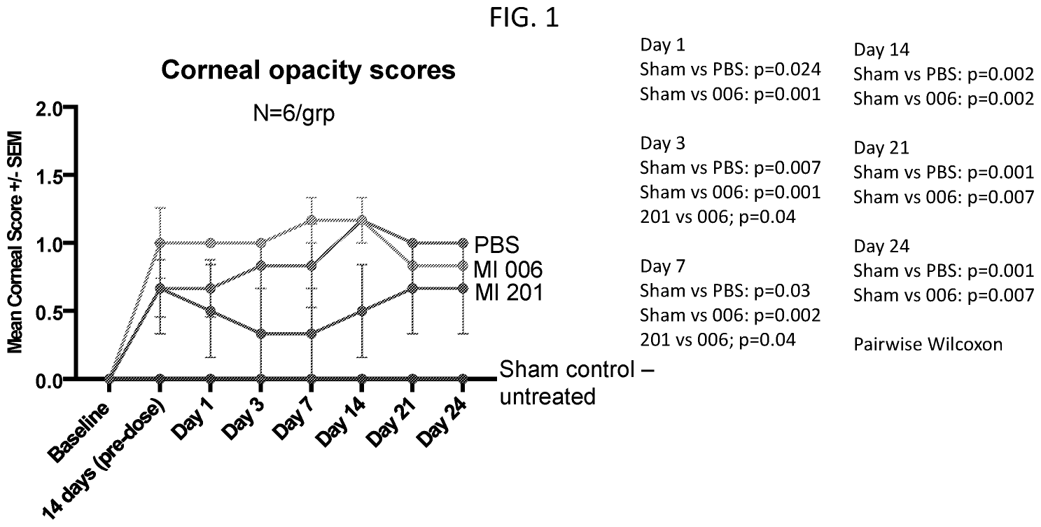

BIO-91201 treated groups exhibited reduced corneal opacity scores relative to the PBS and BIO-11006 dosed groups. For example, at days 3 and 7, Corneal scores were significantly elevated in the PBS and BIO-11006 dosed groups compared to sham, but no difference between sham and BIO-91201 was observed. The clinical opacity scores and statistical analyses are provided in FIG. 1.

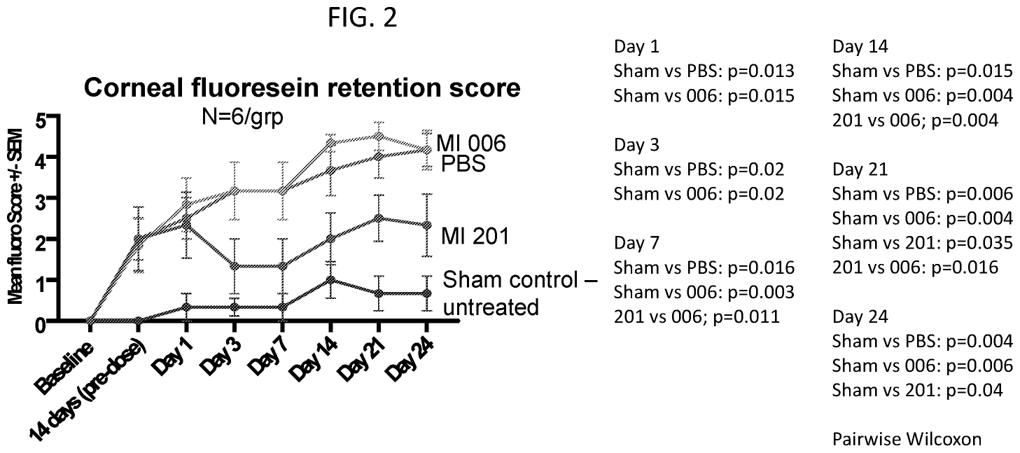

Similarly, BIO-91201 dosed groups had significantly lower FL retention than PBS or BIO-11006 dosed eyes (p<0.02) and through day 14, BIO-91201 FL retention scores were not significantly different than sham eyes. PRT was higher in MARCKs inhibitor-dosed groups compared to PBS groups on days 14, 21, and 24 after surgery (p<0.02; t test). Fluorescein retention score was measured as shown in the table below, and the scores and statistical analyses are provided in FIG. 2.

Fluorescein retention scores.

| Corneal Surface Area | | Density of Damaged Lesions | |
|---|---|---|---|
| A0 | No punctate staining | D0 | No punctate staining |
| A1 | Less than one-third | D1 | Sparse density |
| A2 | One-third to two-thirds | D2 | Moderate density |
| A3 | More than two-thirds | D3 | High density with |
| | | | overlapping lesion |

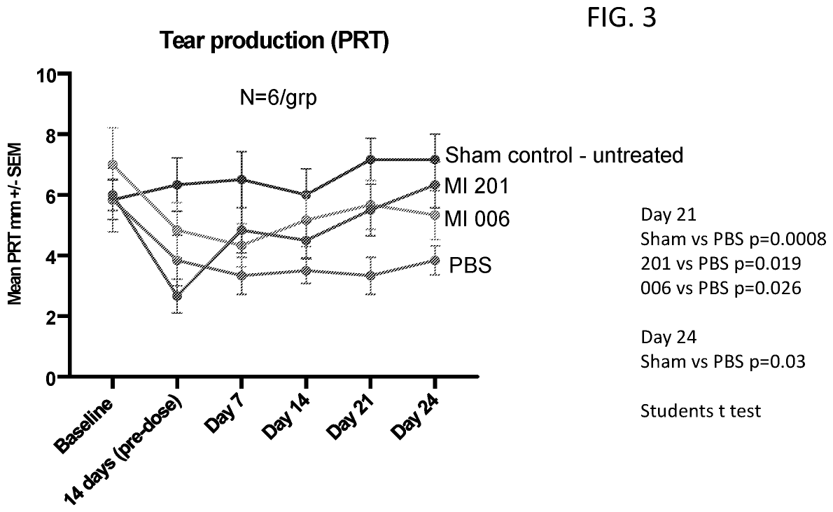

FIG. 3 provides the results of the phenol red test (PRT), showing tear measurement in mm/20 s averaged for each group.

Taken together, the results of the study showed that topical MARCKS inhibitor peptides appear well tolerated and may reduce inflammation, corneal opacity, corneal FL retention, and improve tear production in dry eye. In particular, the smaller peptide, BIO-91201, exhibited a significant positive effect on indicators of dry eye disease including corneal scores and corneal fluorescein retention, as well as improvement in tear production. Both peptides were well tolerated without signs of inflammation. Further development of topical MARCKs inhibitors, especially the smaller peptide, BIO-91201, for treatment of dry eye appears warranted.

The foregoing examples are illustrative of the present disclosure and are not to be construed as limiting thereof. The disclosure is defined by the following claims, with equivalents of the claims to be included therein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

1. A method of treating dry eye syndrome in a subject comprising:
administering to said subject a therapeutically effective amount of a composition comprising at least one peptide having an amino acid sequence consisting of from 4 to 24 contiguous amino acids of a reference sequence, GAQFSKTAAKGEAAAERPGEAAVA (SEQ ID NO. 1).

2. The method of claim 1, wherein said peptide had an amino acid sequence consisting of four contiguous amino acid residues of SEQ ID NO: 1.

3. The method of claim 1, wherein said peptide comprises an amino acid sequence of SEQ ID NO: 106 or 219.

4. The method of any one of claims 1-3, wherein said peptide comprises an amino acid sequence of SEQ ID NO: 219.

5. The method of any one of claims 1-4, wherein said peptide consists of an amino acid sequence of SEQ ID NO: 219.

6. The method of any of claims 1-5, wherein the peptide is myristoylated or acetylated at the N-terminal amino acid.

7. The method of any one of claims 1-6, wherein the peptide is acetylated at the N-terminal amino acid, and consists of an amino acid sequence of SEQ ID NO: 219.

8. The method according to any one of claims 1-7, wherein the composition comprises a pharmaceutically acceptable carrier.

9. The method according to claim 1, wherein said subject is a mammal.

10. The method according to claim 9, wherein said mammal is selected from the group consisting of humans, canines, equines and felines.

11. The method according to any one of claims 1-10, wherein the composition is administered by topical administration, intravitreal injection (IVT), subconjuctival injection, subtenon injection (SBT), retrobulbar injection, periocular injection, subretinal injection, intrascleral, transscleral, intrastromal, intravenous injection, intra-ocular administration, or any combination thereof.

12. The method according to any one of claims 1-11, wherein the composition is administered by topical administration to the eye.

13. The method of claim 11 or 12, wherein the topical administration comprises one, two, three, four, five, or six topical administrations daily.

14. The method according to claim 1, wherein the composition comprises a topical formulation, intra-ocular formulation, an eye implant, an eye drop, eye gel, ointment, microspheres, microemulsion, liposomal formulation or any combination thereof.

15. The method according to claim 1, further comprising administration to said subject a second molecule, wherein the second molecule is an antibiotic, an antiviral compound, an antiparasitic compound, an anti-inflammatory compound, an immunomodulatory compound, or any combination thereof.

16. The method of any one of claims 1-15, wherein the composition is administered at a concentration from about 1 μM to about 1 mM.

17. The method of any one of claims 1-16, wherein the composition is administered in an amount of about 100 μM.

18. The method of any one of claims 1-17, wherein the composition is administered in a volume of about 0.01 mL to about 1 mL.


SEQUENCE LISTING (252 sequences)

SEQ ID NO: 1 - protein, 24 aa, synthetic construct
  SITE 1: note = peptide may or may not be N-term modified
  SITE 24: note = peptide may or may not be C-term modified
GAQFSKTAAKGEAAAERPGEAAVA

SEQ ID NO: 2 - protein, 23 aa, synthetic construct
  SITE 1: note = peptide may or may not be N-term modified
  SITE 23: note = peptide may or may not be C-term modified
GAQFSKTAAKGEAAAERPGEAAV

SEQ ID NO: 3 - protein, 23 aa, synthetic construct
  SITE 1: note = peptide may or may not be N-term modified
  SITE 23: note = peptide may or may not be C-term modified
AQFSKTAAKGEAAAERPGEAAVA

SEQ ID NO: 4 - protein, 22 aa, synthetic construct
  SITE 1: note = peptide may or may not be N-term modified
  SITE 22: note = peptide may or may not be C-term modified
GAQFSKTAAKGEAAAERPGEAA

SEQ ID NO: 5 - protein, 22 aa, synthetic construct
  SITE 1: note = peptide may or may not be N-term modified
  SITE 22: note = peptide may or may not be C-term modified
AQFSKTAAKGEAAAERPGEAAV

SEQ ID NO: 6 - protein, 22 aa, synthetic construct
  SITE 1: note = peptide may or may not be N-term modified
  SITE 22: note = peptide may or may not be C-term modified
QFSKTAAKGEAAAERPGEAAVA

SEQ ID NO: 7 - protein, 21 aa, synthetic construct
  SITE 1: note = peptide may or may not be N-term modified
  SITE 21: note = peptide may or may not be C-term modified
GAQFSKTAAKGEAAAERPGEA

SEQ ID NO: 8 - protein, 21 aa, synthetic construct
  SITE 1: note = peptide may or may not be N-term modified
  SITE 21: note = peptide may or may not be C-term modified
AQFSKTAAKGEAAAERPGEAA

SEQ ID NO: 9 - protein, 21 aa, synthetic construct
  SITE 1: note = peptide may or may not be N-term modified
  SITE 21: note = peptide may or may not be C-term modified
QFSKTAAKGEAAAERPGEAAV

SEQ ID NO: 10 - protein, 21 aa, synthetic construct
  SITE 1: note = peptide may or may not be N-term modified
  SITE 21: note = peptide may or may not be C-term modified
FSKTAAKGEAAAERPGEAAVA

SEQ ID NO: 11 - protein, 20 aa, synthetic construct
  SITE 1: note = peptide may or may not be N-term modified
  SITE 20: note = peptide may or may not be C-term modified
GAQFSKTAAKGEAAAERPGE

SEQ ID NO: 12 - protein, 20 aa, synthetic construct
  SITE 1: note = peptide may or may not be N-term modified
  SITE 20: note = peptide may or may not be C-term modified
AQFSKTAAKGEAAAERPGEA

SEQ ID NO: 13 - protein, 20 aa, synthetic construct
  SITE 1: note = peptide may or may not be N-term modified
  SITE 20: note = peptide may or may not be C-term modified
QFSKTAAKGEAAAERPGEAA

SEQ ID NO: 14 - protein, 20 aa, synthetic construct
  SITE 1: note = peptide may or may not be N-term modified
  SITE 20: note = peptide may or may not be C-term modified
FSKTAAKGEAAAERPGEAAV

SEQ ID NO: 15 - protein, 20 aa, synthetic construct
  SITE 1: note = peptide may or may not be N-term modified
  SITE 20: note = peptide may or may not be C-term modified
SKTAAKGEAAAERPGEAAVA

SEQ ID NO: 16 - protein, 19 aa, synthetic construct
  SITE 1: note = peptide may or may not be N-term modified
  SITE 19: note = peptide may or may not be C-term modified
GAQFSKTAAKGEAAAERPG

SEQ ID NO: 17 - protein, 19 aa, synthetic construct
  SITE 1: note = peptide may or may not be N-term modified
  SITE 19: note = peptide may or may not be C-term modified
AQFSKTAAKGEAAAERPGE

SEQ ID NO: 18 - protein, 19 aa, synthetic construct
  SITE 1: note = peptide may or may not be N-term modified
  SITE 19: note = peptide may or may not be C-term modified
QFSKTAAKGEAAAERPGEA

SEQ ID NO: 19 - protein, 19 aa, synthetic construct
  SITE 1: note = peptide may or may not be N-term modified
  SITE 19: note = peptide may or may not be C-term modified
FSKTAAKGEAAAERPGEAA

SEQ ID NO: 20 - protein, 19 aa, synthetic construct
  SITE 1: note = peptide may or may not be N-term modified
  SITE 19: note = peptide may or may not be C-term modified
SKTAAKGEAAAERPGEAAV

SEQ ID NO: 21 - protein, 19 aa, synthetic construct
  SITE 1: note = peptide may or may not be N-term modified
  SITE 19: note = peptide may or may not be C-term modified
KTAAKGEAAAERPGEAAVA

SEQ ID NO: 22 - protein, 18 aa, synthetic construct
  SITE 1: note = peptide may or may not be N-term modified
  SITE 18: note = peptide may or may not be C-term modified
GAQFSKTAAKGEAAAERP

SEQ ID NO: 23 - protein, 18 aa, synthetic construct
  SITE 1: note = peptide may or may not be N-term modified
  SITE 18: note = peptide may or may not be C-term modified
AQFSKTAAKGEAAAERPG

SEQ ID NO: 24 - protein, 18 aa, synthetic construct
  SITE 1: note = peptide may or may not be N-term modified
  SITE 18: note = peptide may or may not be C-term modified
QFSKTAAKGEAAAERPGE

SEQ ID NO: 25 - protein, 18 aa, synthetic construct
  SITE 1: note = peptide may or may not be N-term modified
  SITE 18: note = peptide may or may not be C-term modified
FSKTAAKGEAAAERPGEA

SEQ ID NO: 26 - protein, 18 aa, synthetic construct
  SITE 1: note = peptide may or may not be N-term modified
  SITE 18: note = peptide may or may not be C-term modified
SKTAAKGEAAAERPGEAA

SEQ ID NO: 27 - protein, 18 aa, synthetic construct
  SITE 1: note = peptide may or may not be N-term modified
  SITE 18: note = peptide may or may not be C-term modified
KTAAKGEAAAERPGEAAV

SEQ ID NO: 28 - protein, 18 aa, synthetic construct
  SITE 1: note = peptide may or may not be N-term modified
  SITE 18: note = peptide may or may not be C-term modified
TAAKGEAAAERPGEAAVA

SEQ ID NO: 29 - protein, 17 aa, synthetic construct
  SITE 1: note = peptide may or may not be N-term modified
  SITE 17: note = peptide may or may not be C-term modified
GAQFSKTAAKGEAAAER

SEQ ID NO: 30 - protein, 17 aa, synthetic construct
  SITE 1: note = peptide may or may not be N-term modified
  SITE 17: note = peptide may or may not be C-term modified
AQFSKTAAKGEAAAERP

SEQ ID NO: 31 - protein, 17 aa, synthetic construct
  SITE 1: note = peptide may or may not be N-term modified
  SITE 17: note = peptide may or may not be C-term modified
QFSKTAAKGEAAAERPG

SEQ ID NO: 32 - protein, 17 aa, synthetic construct
  SITE 1: note = peptide may or may not be N-term modified
  SITE 17: note = peptide may or may not be C-term modified
FSKTAAKGEAAAERPGE

SEQ ID NO: 33 - protein, 17 aa, synthetic construct
  SITE 1: note = peptide may or may not be N-term modified
  SITE 17: note = peptide may or may not be C-term modified
SKTAAKGEAAAERPGEA

SEQ ID NO: 34 - protein, 17 aa, synthetic construct
  SITE 1: note = peptide may or may not be N-term modified
  SITE 17: note = peptide may or may not be C-term modified
KTAAKGEAAAERPGEAA

SEQ ID NO: 35 - protein, 17 aa, synthetic construct
  SITE 1: note = peptide may or may not be N-term modified
  SITE 17: note = peptide may or may not be C-term modified
TAAKGEAAAERPGEAAV

SEQ ID NO: 36 - protein, 17 aa, synthetic construct
  SITE 1: note = peptide may or may not be N-term modified
  SITE 17: note = peptide may or may not be C-term modified
AAKGEAAAERPGEAAVA

SEQ ID NO: 37 - protein, 16 aa, synthetic construct
  SITE 1: note = peptide may or may not be N-term modified
  SITE 16: note = peptide may or may not be C-term modified
GAQFSKTAAKGEAAAE

SEQ ID NO: 38 - protein, 16 aa, synthetic construct
  SITE 1: note = peptide may or may not be N-term modified
  SITE 16: note = peptide may or may not be C-term modified
AQFSKTAAKGEAAAER

SEQ ID NO: 39 - protein, 16 aa, synthetic construct
  SITE 1: note = peptide may or may not be N-term modified
  SITE 16: note = peptide may or may not be C-term modified
QFSKTAAKGEAAAERP

SEQ ID NO: 40 - protein, 16 aa, synthetic construct
  SITE 1: note = peptide may or may not be N-term modified
  SITE 16: note = peptide may or may not be C-term modified
FSKTAAKGEAAAERPG

SEQ ID NO: 41 - protein, 16 aa, synthetic construct
  SITE 1: note = peptide may or may not be N-term modified
  SITE 16: note = peptide may or may not be C-term modified
SKTAAKGEAAAERPGE

SEQ ID NO: 42 - protein, 16 aa, synthetic construct
  SITE 1: note = peptide may or may not be N-term modified
  SITE 16: note = peptide may or may not be C-term modified
KTAAKGEAAAERPGEA

SEQ ID NO: 43 - protein, 16 aa, synthetic construct
  SITE 1: note = peptide may or may not be N-term modified
  SITE 16: note = peptide may or may not be C-term modified
TAAKGEAAAERPGEAA

SEQ ID NO: 44 - protein, 16 aa, synthetic construct
  SITE 1: note = peptide may or may not be N-term modified
  SITE 16: note = peptide may or may not be C-term modified
AAKGEAAAERPGEAAV

SEQ ID NO: 45 - protein, 16 aa, synthetic construct
  SITE 1: note = peptide may or may not be N-term modified
  SITE 16: note = peptide may or may not be C-term modified
AKGEAAAERPGEAAVA

SEQ ID NO: 46 - protein, 15 aa, synthetic construct
  SITE 1: note = peptide may or may not be N-term modified
  SITE 15: note = peptide may or may not be C-term modified
GAQFSKTAAKGEAAA

SEQ ID NO: 47 - protein, 15 aa, synthetic construct
  SITE 1: note = peptide may or may not be N-term modified
  SITE 15: note = peptide may or may not be C-term modified
AQFSKTAAKGEAAAE

SEQ ID NO: 48 - protein, 15 aa, synthetic construct
  SITE 1: note = peptide may or may not be N-term modified
  SITE 15: note = peptide may or may not be C-term modified
QFSKTAAKGEAAAER

SEQ ID NO: 49 - protein, 15 aa, synthetic construct
  SITE 1: note = peptide may or may not be N-term modified
  SITE 15: note = peptide may or may not be C-term modified
FSKTAAKGEAAAERP

SEQ ID NO: 50 - protein, 15 aa, synthetic construct
  SITE 1: note = peptide may or may not be N-term modified
  SITE 15: note = peptide may or may not be C-term modified
SKTAAKGEAAAERPG

SEQ ID NO: 51 - protein, 15 aa, synthetic construct
  SITE 1: note = peptide may or may not be N-term modified
  SITE 15: note = peptide may or may not be C-term modified
KTAAKGEAAAERPGE

SEQ ID NO: 52 - protein, 15 aa, synthetic construct
  SITE 1: note = peptide may or may not be N-term modified
  SITE 15: note = peptide may or may not be C-term modified
TAAKGEAAAERPGEA

SEQ ID NO: 53 - protein, 15 aa, synthetic construct
  SITE 1: note = peptide may or may not be N-term modified
  SITE 15: note = peptide may or may not be C-term modified
AAKGEAAAERPGEAA

SEQ ID NO: 54 - protein, 15 aa, synthetic construct
  SITE 1: note = peptide may or may not be N-term modified
  SITE 15: note = peptide may or may not be C-term modified
AKGEAAAERPGEAAV

SEQ ID NO: 55 - protein, 15 aa, synthetic construct
  SITE 1: note = peptide may or may not be N-term modified
  SITE 15: note = peptide may or may not be C-term modified
KGEAAAERPGEAAVA

SEQ ID NO: 56 - protein, 14 aa, synthetic construct
  SITE 1: note = peptide may or may not be N-term modified
  SITE 14: note = peptide may or may not be C-term modified
GAQFSKTAAKGEAA

SEQ ID NO: 57 - protein, 14 aa, synthetic construct
  SITE 1: note = peptide may or may not be N-term modified
  SITE 14: note = peptide may or may not be C-term modified
AQFSKTAAKGEAAA

SEQ ID NO: 58 - protein, 14 aa, synthetic construct
  SITE 1: note = peptide may or may not be N-term modified
  SITE 14: note = peptide may or may not be C-term modified
QFSKTAAKGEAAAE

SEQ ID NO: 59 - protein, 14 aa, synthetic construct
  SITE 1: note = peptide may or may not be N-term modified
  SITE 14: note = peptide may or may not be C-term modified
FSKTAAKGEAAAER

SEQ ID NO: 60 - protein, 14 aa, synthetic construct
  SITE 1: note = peptide may or may not be N-term modified
  SITE 14: note = peptide may or may not be C-term modified
SKTAAKGEAAAERP

SEQ ID NO: 61 - protein, 14 aa, synthetic construct
  SITE 1: note = peptide may or may not be N-term modified
  SITE 14: note = peptide may or may not be C-term modified
KTAAKGEAAAERPG

SEQ ID NO: 62 - protein, 14 aa, synthetic construct
  SITE 1: note = peptide may or may not be N-term modified
  SITE 14: note = peptide may or may not be C-term modified
TAAKGEAAAERPGE

SEQ ID NO: 63 - protein, 14 aa, synthetic construct
  SITE 1: note = peptide may or may not be N-term modified
  SITE 14: note = peptide may or may not be C-term modified
AAKGEAAAERPGEA

SEQ ID NO: 64 - protein, 14 aa, synthetic construct
  SITE 1: note = peptide may or may not be N-term modified
  SITE 14: note = peptide may or may not be C-term modified
AKGEAAAERPGEAA

SEQ ID NO: 65 - protein, 14 aa, synthetic construct
  SITE 1: note = peptide may or may not be N-term modified
  SITE 14: note = peptide may or may not be C-term modified
KGEAAAERPGEAAV

SEQ ID NO: 66 - protein, 14 aa, synthetic construct
  SITE 1: note = peptide may or may not be N-term modified
  SITE 14: note = peptide may or may not be C-term modified
GEAAAERPGEAAVA

SEQ ID NO: 67 - protein, 13 aa, synthetic construct
  SITE 1: note = peptide may or may not be N-term modified
  SITE 13: note = peptide may or may not be C-term modified
GAQFSKTAAKGEA

SEQ ID NO: 68 - protein, 13 aa, synthetic construct
  SITE 1: note = peptide may or may not be N-term modified
  SITE 13: note = peptide may or may not be C-term modified
AQFSKTAAKGEAA

SEQ ID NO: 69 - protein, 13 aa, synthetic construct
  SITE 1: note = peptide may or may not be N-term modified
  SITE 13: note = peptide may or may not be C-term modified
QFSKTAAKGEAAA

SEQ ID NO: 70 - protein, 13 aa, synthetic construct
  SITE 1: note = peptide may or may not be N-term modified
  SITE 13: note = peptide may or may not be C-term modified
FSKTAAKGEAAAE

SEQ ID NO: 71 - protein, 13 aa, synthetic construct
  SITE 1: note = peptide may or may not be N-term modified
  SITE 13: note = peptide may or may not be C-term modified
SKTAAKGEAAAER

SEQ ID NO: 72 - protein, 13 aa, synthetic construct
  SITE 1: note = peptide may or may not be N-term modified
  SITE 13: note = peptide may or may not be C-term modified
KTAAKGEAAAERP

SEQ ID NO: 73 - protein, 13 aa, synthetic construct
  SITE 1: note = peptide may or may not be N-term modified
  SITE 13: note = peptide may or may not be C-term modified
TAAKGEAAAERPG

SEQ ID NO: 74 - protein, 13 aa, synthetic construct
  SITE 1: note = peptide may or may not be N-term modified
  SITE 13: note = peptide may or may not be C-term modified
AAKGEAAAERPGE

SEQ ID NO: 75 - protein, 13 aa, synthetic construct
  SITE 1: note = peptide may or may not be N-term modified
  SITE 13: note = peptide may or may not be C-term modified
AKGEAAAERPGEA

SEQ ID NO: 76 - protein, 13 aa, synthetic construct
  SITE 1: note = peptide may or may not be N-term modified
  SITE 13: note = peptide may or may not be C-term modified
KGEAAAERPGEAA

SEQ ID NO: 77 - protein, 13 aa, synthetic construct
  SITE 1: note = peptide may or may not be N-term modified
  SITE 13: note = peptide may or may not be C-term modified
GEAAAERPGEAAV

SEQ ID NO: 78 - protein, 13 aa, synthetic construct
  SITE 1: note = peptide may or may not be N-term modified
  SITE 13: note = peptide may or may not be C-term modified
EAAAERPGEAAVA

SEQ ID NO: 79 - protein, 12 aa, synthetic construct
  SITE 1: note = peptide may or may not be N-term modified
  SITE 12: note = peptide may or may not be C-term modified
GAQFSKTAAKGE

SEQ ID NO: 80 - protein, 12 aa, synthetic construct
  SITE 1: note = peptide may or may not be N-term modified
  SITE 12: note = peptide may or may not be C-term modified
AQFSKTAAKGEA

SEQ ID NO: 81 - protein, 12 aa, synthetic construct
  SITE 1: note = peptide may or may not be N-term modified
  SITE 12: note = peptide may or may not be C-term modified
QFSKTAAKGEAA

SEQ ID NO: 82 - protein, 12 aa, synthetic construct
  SITE 1: note = peptide may or may not be N-term modified
  SITE 12: note = peptide may or may not be C-term modified
FSKTAAKGEAAA

SEQ ID NO: 83 - protein, 12 aa, synthetic construct
  SITE 1: note = peptide may or may not be N-term modified
  SITE 12: note = peptide may or may not be C-term modified
SKTAAKGEAAAE

SEQ ID NO: 84 - protein, 12 aa, synthetic construct
  SITE 1: note = peptide may or may not be N-term modified
  SITE 12: note = peptide may or may not be C-term modified
KTAAKGEAAAER

SEQ ID NO: 85 - protein, 12 aa, synthetic construct
  SITE 1: note = peptide may or may not be N-term modified
  SITE 12: note = peptide may or may not be C-term modified
TAAKGEAAAERP

SEQ ID NO: 86 - protein, 12 aa, synthetic construct
  SITE 1: note = peptide may or may not be N-term modified
  SITE 12: note = peptide may or may not be C-term modified
AAKGEAAAERPG

SEQ ID NO: 87 - protein, 12 aa, synthetic construct
  SITE 1: note = peptide may or may not be N-term modified
  SITE 12: note = peptide may or may not be C-term modified
AKGEAAAERPGE

SEQ ID NO: 88 - protein, 12 aa, synthetic construct
  SITE 1: note = peptide may or may not be N-term modified
  SITE 12: note = peptide may or may not be C-term modified
KGEAAAERPGEA

SEQ ID NO: 89 - protein, 12 aa, synthetic construct
  SITE 1: note = peptide may or may not be N-term modified
  SITE 12: note = peptide may or may not be C-term modified
GEAAAERPGEAA

SEQ ID NO: 90 - protein, 12 aa, synthetic construct
  SITE 1: note = peptide may or may not be N-term modified
  SITE 12: note = peptide may or may not be C-term modified
EAAAERPGEAAV

SEQ ID NO: 91 - protein, 12 aa, synthetic construct
  SITE 1: note = peptide may or may not be N-term modified
  SITE 12: note = peptide may or may not be C-term modified
AAAERPGEAAVA

SEQ ID NO: 92 - protein, 11 aa, synthetic construct
  SITE 1: note = peptide may or may not be N-term modified
  SITE 11: note = peptide may or may not be C-term modified
GAQFSKTAAKG

SEQ ID NO: 93 - protein, 11 aa, synthetic construct
  SITE 1: note = peptide may or may not be N-term modified
  SITE 11: note = peptide may or may not be C-term modified
AQFSKTAAKGE

SEQ ID NO: 94 - protein, 11 aa, synthetic construct
  SITE 1: note = peptide may or may not be N-term modified
  SITE 11: note = peptide may or may not be C-term modified
QFSKTAAKGEA

SEQ ID NO: 95 - protein, 11 aa, synthetic construct
  SITE 1: note = peptide may or may not be N-term modified
  SITE 11: note = peptide may or may not be C-term modified
FSKTAAKGEAA

SEQ ID NO: 96 - protein, 11 aa, synthetic construct
  SITE 1: note = peptide may or may not be N-term modified
  SITE 11: note = peptide may or may not be C-term modified
SKTAAKGEAAA

SEQ ID NO: 97 - protein, 11 aa, synthetic construct
  SITE 1: note = peptide may or may not be N-term modified
  SITE 11: note = peptide may or may not be C-term modified
KTAAKGEAAAE

SEQ ID NO: 98 - protein, 11 aa, synthetic construct
  SITE 1: note = peptide may or may not be N-term modified
  SITE 11: note = peptide may or may not be C-term modified
TAAKGEAAAER

SEQ ID NO: 99 - protein, 11 aa, synthetic construct
  SITE 1: note = peptide may or may not be N-term modified
  SITE 11: note = peptide may or may not be C-term modified
AAKGEAAAERP

SEQ ID NO: 100 - protein, 11 aa, synthetic construct
  SITE 1: note = peptide may or may not be N-term modified
  SITE 11: note = peptide may or may not be C-term modified
AKGEAAAERPG

SEQ ID NO: 101 - protein, 11 aa, synthetic construct
  SITE 1: note = peptide may or may not be N-term modified
  SITE 11: note = peptide may or may not be C-term modified
KGEAAAERPGE

SEQ ID NO: 102 - protein, 11 aa, synthetic construct
  SITE 1: note = peptide may or may not be N-term modified
  SITE 11: note = peptide may or may not be C-term modified
GEAAAERPGEA

SEQ ID NO: 103 - protein, 11 aa, synthetic construct
  SITE 1: note = peptide may or may not be N-term modified
  SITE 11: note = peptide may or may not be C-term modified
EAAAERPGEAA

SEQ ID NO: 104 - protein, 11 aa, synthetic construct
  SITE 1: note = peptide may or may not be N-term modified
  SITE 11: note = peptide may or may not be C-term modified
AAAERPGEAAV

SEQ ID NO: 105 - protein, 11 aa, synthetic construct
  SITE 1: note = peptide may or may not be N-term modified
  SITE 11: note = peptide may or may not be C-term modified
AAERPGEAAVA

SEQ ID NO: 106 - protein, 10 aa, synthetic construct
  SITE 1: note = peptide may or may not be N-term modified
  SITE 10: note = peptide may or may not be C-term modified
GAQFSKTAAK

SEQ ID NO: 107 - protein, 10 aa, synthetic construct
  SITE 1: note = peptide may or may not be N-term modified
  SITE 10: note = peptide may or may not be C-term modified
AQFSKTAAKG

SEQ ID NO: 108 - protein, 10 aa, synthetic construct
  SITE 1: note = peptide may or may not be N-term modified
  SITE 10: note = peptide may or may not be C-term modified
QFSKTAAKGE

SEQ ID NO: 109 - protein, 10 aa, synthetic construct
  SITE 1: note = peptide may or may not be N-term modified
  SITE 10: note = peptide may or may not be C-term modified
FSKTAAKGEA

SEQ ID NO: 110 - protein, 10 aa, synthetic construct
  SITE 1: note = peptide may or may not be N-term modified
  SITE 10: note = peptide may or may not be C-term modified
SKTAAKGEAA

SEQ ID NO: 111 - protein, 10 aa, synthetic construct
  SITE 1: note = peptide may or may not be N-term modified
  SITE 10: note = peptide may or may not be C-term modified
KTAAKGEAAA

SEQ ID NO: 112 - protein, 10 aa, synthetic construct
  SITE 1: note = peptide may or may not be N-term modified
  SITE 10: note = peptide may or may not be C-term modified
TAAKGEAAAE

SEQ ID NO: 113 - protein, 10 aa, synthetic construct
  SITE 1: note = peptide may or may not be N-term modified
  SITE 10: note = peptide may or may not be C-term modified
AAKGEAAAER

SEQ ID NO: 114 - protein, 10 aa, synthetic construct
  SITE 1: note = peptide may or may not be N-term modified
  SITE 10: note = peptide may or may not be C-term modified
AKGEAAAERP

SEQ ID NO: 115 - protein, 10 aa, synthetic construct
  SITE 1: note = peptide may or may not be N-term modified
  SITE 10: note = peptide may or may not be C-term modified
KGEAAAERPG

SEQ ID NO: 116 - protein, 10 aa, synthetic construct
  SITE 1: note = peptide may or may not be N-term modified
  SITE 10: note = peptide may or may not be C-term modified
GEAAAERPGE

SEQ ID NO: 117 - protein, 10 aa, synthetic construct
  SITE 1: note = peptide may or may not be N-term modified
  SITE 10: note = peptide may or may not be C-term modified
EAAAERPGEA

SEQ ID NO: 118 - protein, 10 aa, synthetic construct
  SITE 1: note = peptide may or may not be N-term modified
  SITE 10: note = peptide may or may not be C-term modified
AAAERPGEAA

SEQ ID NO: 119 - protein, 10 aa, synthetic construct
  SITE 1: note = peptide may or may not be N-term modified
  SITE 10: note = peptide may or may not be C-term modified
AAERPGEAAV

SEQ ID NO: 120 - protein, 10 aa, synthetic construct
  SITE 1: note = peptide may or may not be N-term modified
  SITE 10: note = peptide may or may not be C-term modified
AERPGEAAVA

SEQ ID NO: 121 - protein, 9 aa, synthetic construct
  SITE 1: note = peptide may or may not be N-term modified
  SITE 9: note = peptide may or may not be C-term modified
GAQFSKTAA

SEQ ID NO: 122 - protein, 9 aa, synthetic construct
  SITE 1: note = peptide may or may not be N-term modified
  SITE 9: note = peptide may or may not be C-term modified
AQFSKTAAK

SEQ ID NO: 123 - protein, 9 aa, synthetic construct
  SITE 1: note = peptide may or may not be N-term modified
  SITE 9: note = peptide may or may not be C-term modified
QFSKTAAKG

SEQ ID NO: 124 - protein, 9 aa, synthetic construct
  SITE 1: note = peptide may or may not be N-term modified
  SITE 9: note = peptide may or may not be C-term modified
FSKTAAKGE

SEQ ID NO: 125 - protein, 9 aa, synthetic construct
  SITE 1: note = peptide may or may not be N-term modified
  SITE 9: note = peptide may or may not be C-term modified
SKTAAKGEA

SEQ ID NO: 126 - protein, 9 aa, synthetic construct
  SITE 1: note = peptide may or may not be N-term modified
  SITE 9: note = peptide may or may not be C-term modified
KTAAKGEAA

SEQ ID NO: 127 - protein, 9 aa, synthetic construct
  SITE 1: note = peptide may or may not be N-term modified
  SITE 9: note = peptide may or may not be C-term modified
TAAKGEAAA

SEQ ID NO: 128 - protein, 9 aa, synthetic construct
  SITE 1: note = peptide may or may not be N-term modified
  SITE 9: note = peptide may or may not be C-term modified
AAKGEAAAE

SEQ ID NO: 129 - protein, 9 aa, synthetic construct
  SITE 1: note = peptide may or may not be N-term modified
  SITE 9: note = peptide may or may not be C-term modified
AKGEAAAER

SEQ ID NO: 130 - protein, 9 aa, synthetic construct
  SITE 1: note = peptide may or may not be N-term modified
  SITE 9: note = peptide may or may not be C-term modified
KGEAAAERP

SEQ ID NO: 131 - protein, 9 aa, synthetic construct
  SITE 1: note = peptide may or may not be N-term modified
  SITE 9: note = peptide may or may not be C-term modified
GEAAAERPG

SEQ ID NO: 132 - protein, 9 aa, synthetic construct
  SITE 1: note = peptide may or may not be N-term modified
  SITE 9: note = peptide may or may not be C-term modified
EAAAERPGE

SEQ ID NO: 133 - protein, 9 aa, synthetic construct
  SITE 1: note = peptide may or may not be N-term modified
  SITE 9: note = peptide may or may not be C-term modified
AAAERPGEA

SEQ ID NO: 134 - protein, 9 aa, synthetic construct
  SITE 1: note = peptide may or may not be N-term modified
  SITE 9: note = peptide may or may not be C-term modified
AAERPGEAA

SEQ ID NO: 135 - protein, 9 aa, synthetic construct
  SITE 1: note = peptide may or may not be N-term modified
  SITE 9: note = peptide may or may not be C-term modified
AERPGEAAV

SEQ ID NO: 136 - protein, 9 aa, synthetic construct
  SITE 1: note = peptide may or may not be N-term modified
  SITE 9: note = peptide may or may not be C-term modified
ERPGEAAVA

SEQ ID NO: 137 - protein, 8 aa, synthetic construct
  SITE 1: note = peptide may or may not be N-term modified
  SITE 8: note = peptide may or may not be C-term modified
GAQFSKTA

SEQ ID NO: 138 - protein, 8 aa, synthetic construct
  SITE 1: note = peptide may or may not be N-term modified
  SITE 8: note = peptide may or may not be C-term modified
AQFSKTAA

SEQ ID NO: 139 - protein, 8 aa, synthetic construct
  SITE 1: note = peptide may or may not be N-term modified
  SITE 8: note = peptide may or may not be C-term modified
QFSKTAAK

SEQ ID NO: 140 - protein, 8 aa, synthetic construct
  SITE 1: note = peptide may or may not be N-term modified
  SITE 8: note = peptide may or may not be C-term modified
FSKTAAKG

SEQ ID NO: 141 - protein, 8 aa, synthetic construct
  SITE 1: note = peptide may or may not be N-term modified
  SITE 8: note = peptide may or may not be C-term modified
SKTAAKGE

SEQ ID NO: 142 - protein, 8 aa, synthetic construct
  SITE 1: note = peptide may or may not be N-term modified
  SITE 8: note = peptide may or may not be C-term modified
KTAAKGEA

SEQ ID NO: 143 - protein, 8 aa, synthetic construct
  SITE 1: note = peptide may or may not be N-term modified
  SITE 8: note = peptide may or may not be C-term modified
TAAKGEAA

SEQ ID NO: 144 - protein, 8 aa, synthetic construct
  SITE 1: note = peptide may or may not be N-term modified
  SITE 8: note = peptide may or may not be C-term modified
AAKGEAAA

SEQ ID NO: 145 - protein, 8 aa, synthetic construct
  SITE 1: note = peptide may or may not be N-term modified
  SITE 8: note = peptide may or may not be C-term modified
AKGEAAAE

SEQ ID NO: 146 - protein, 8 aa, synthetic construct
  SITE 1: note = peptide may or may not be N-term modified
  SITE 8: note = peptide may or may not be C-term modified
KGEAAAER

SEQ ID NO: 147 - protein, 8 aa, synthetic construct
  SITE 1: note = peptide may or may not be N-term modified
  SITE 8: note = peptide may or may not be C-term modified
GEAAAERP

SEQ ID NO: 148 - protein, 8 aa, synthetic construct
  SITE 1: note = peptide may or may not be N-term modified
  SITE 8: note = peptide may or may not be C-term modified
EAAAERPG

SEQ ID NO: 149 - protein, 8 aa, synthetic construct
  SITE 1: note = peptide may or may not be N-term modified
  SITE 8: note = peptide may or may not be C-term modified
AAAERPGE

SEQ ID NO: 150 - protein, 8 aa, synthetic construct
  SITE 1: note = peptide may or may not be N-term modified
  SITE 8: note = peptide may or may not be C-term modified
AAERPGEA

SEQ ID NO: 151 - protein, 8 aa, synthetic construct
  SITE 1: note = peptide may or may not be N-term modified
  SITE 8: note = peptide may or may not be C-term modified
AERPGEAA

SEQ ID NO: 152 - protein, 8 aa, synthetic construct
  SITE 1: note = peptide may or may not be N-term modified
  SITE 8: note = peptide may or may not be C-term modified
ERPGEAAV

SEQ ID NO: 153 - protein, 8 aa, synthetic construct
  SITE 1: note = peptide may or may not be N-term modified
  SITE 8: note = peptide may or may not be C-term modified
RPGEAAVA

SEQ ID NO: 154 - protein, 7 aa, synthetic construct
  SITE 1: note = peptide may or may not be N-term modified
  SITE 7: note = peptide may or may not be C-term modified
GAQFSKT

SEQ ID NO: 155 - protein, 7 aa, synthetic construct
  SITE 1: note = peptide may or may not be N-term modified
  SITE 7: note = peptide may or may not be C-term modified
AQFSKTA

SEQ ID NO: 156 - protein, 7 aa, synthetic construct
  SITE 1: note = peptide may or may not be N-term modified
  SITE 7: note = peptide may or may not be C-term modified
QFSKTAA

SEQ ID NO: 157 - protein, 7 aa, synthetic construct
  SITE 1: note = peptide may or may not be N-term modified
  SITE 7: note = peptide may or may not be C-term modified
FSKTAAK

SEQ ID NO: 158 - protein, 7 aa, synthetic construct
  SITE 1: note = peptide may or may not be N-term modified
  SITE 7: note = peptide may or may not be C-term modified
SKTAAKG

SEQ ID NO: 159 - protein, 7 aa, synthetic construct
  SITE 1: note = peptide may or may not be N-term modified
  SITE 7: note = peptide may or may not be C-term modified
KTAAKGE

SEQ ID NO: 160 - protein, 7 aa, synthetic construct
  SITE 1: note = peptide may or may not be N-term modified
  SITE 7: note = peptide may or may not be C-term modified
TAAKGEA

SEQ ID NO: 161 - protein, 7 aa, synthetic construct
  SITE 1: note = peptide may or may not be N-term modified
  SITE 7: note = peptide may or may not be C-term modified
AAKGEAA

SEQ ID NO: 162 - protein, 7 aa, synthetic construct
  SITE 1: note = peptide may or may not be N-term modified
  SITE 7: note = peptide may or may not be C-term modified
AKGEAAA

SEQ ID NO: 163 - protein, 7 aa, synthetic construct
  SITE 1: note = peptide may or may not be N-term modified
  SITE 7: note = peptide may or may not be C-term modified
KGEAAAE

SEQ ID NO: 164 - protein, 7 aa, synthetic construct
  SITE 1: note = peptide may or may not be N-term modified
  SITE 7: note = peptide may or may not be C-term modified
GEAAAER

SEQ ID NO: 165 - protein, 7 aa, synthetic construct
  SITE 1: note = peptide may or may not be N-term modified
  SITE 7: note = peptide may or may not be C-term modified
EAAAERP

SEQ ID NO: 166 - protein, 7 aa, synthetic construct
  SITE 1: note = peptide may or may not be N-term modified
  SITE 7: note = peptide may or may not be C-term modified
AAAERPG

SEQ ID NO: 167 - protein, 7 aa, synthetic construct
  SITE 1: note = peptide may or may not be N-term modified
  SITE 7: note = peptide may or may not be C-term modified
AAERPGE

SEQ ID NO: 168 - protein, 7 aa, synthetic construct
  SITE 1: note = peptide may or may not be N-term modified
  SITE 7: note = peptide may or may not be C-term modified
AERPGEA

SEQ ID NO: 169 - protein, 7 aa, synthetic construct
  SITE 1: note = peptide may or may not be N-term modified
  SITE 7: note = peptide may or may not be C-term modified
ERPGEAA

SEQ ID NO: 170 - protein, 7 aa, synthetic construct
  SITE 1: note = peptide may or may not be N-term modified
  SITE 7: note = peptide may or may not be C-term modified
RPGEAAV

SEQ ID NO: 171 - protein, 7 aa, synthetic construct
  SITE 1: note = peptide may or may not be N-term modified
  SITE 7: note = peptide may or may not be C-term modified
PGEAAVA

SEQ ID NO: 172 - protein, 6 aa, synthetic construct
  SITE 1: note = peptide may or may not be N-term modified
  SITE 6: note = peptide may or may not be C-term modified
GAQFSK

SEQ ID NO: 173 - protein, 6 aa, synthetic construct
  SITE 1: note = peptide may or may not be N-term modified
  SITE 6: note = peptide may or may not be C-term modified
AQFSKT

SEQ ID NO: 174 - protein, 6 aa, synthetic construct
  SITE 1: note = peptide may or may not be N-term modified
  SITE 6: note = peptide may or may not be C-term modified
QFSKTA

SEQ ID NO: 175 - protein, 6 aa, synthetic construct
  SITE 1: note = peptide may or may not be N-term modified
  SITE 6: note = peptide may or may not be C-term modified
FSKTAA

SEQ ID NO: 176 - protein, 6 aa, synthetic construct
  SITE 1: note = peptide may or may not be N-term modified
  SITE 6: note = peptide may or may not be C-term modified
SKTAAK

SEQ ID NO: 177 - protein, 6 aa, synthetic construct
  SITE 1: note = peptide may or may not be N-term modified
  SITE 6: note = peptide may or may not be C-term modified
KTAAKG

SEQ ID NO: 178 - protein, 6 aa, synthetic construct
  SITE 1: note = peptide may or may not be N-term modified
  SITE 6: note = peptide may or may not be C-term modified
TAAKGE

SEQ ID NO: 179 - protein, 6 aa, synthetic construct
  SITE 1: note = peptide may or may not be N-term modified
  SITE 6: note = peptide may or may not be C-term modified
AAKGEA

SEQ ID NO: 180 - protein, 6 aa, synthetic construct
  SITE 1: note = peptide may or may not be N-term modified
  SITE 6: note = peptide may or may not be C-term modified
AKGEAA

SEQ ID NO: 181 - protein, 6 aa, synthetic construct
  SITE 1: note = peptide may or may not be N-term modified
  SITE 6: note = peptide may or may not be C-term modified
KGEAAA

SEQ ID NO: 182 - protein, 6 aa, synthetic construct
  SITE 1: note = peptide may or may not be N-term modified
  SITE 6: note = peptide may or may not be C-term modified
GEAAAE

SEQ ID NO: 183 - protein, 6 aa, synthetic construct
  SITE 1: note = peptide may or may not be N-term modified
  SITE 6: note = peptide may or may not be C-term modified
EAAAER

SEQ ID NO: 184 - protein, 6 aa, synthetic construct
  SITE 1: note = peptide may or may not be N-term modified
  SITE 6: note = peptide may or may not be C-term modified
AAAERP

SEQ ID NO: 185 - protein, 6 aa, synthetic construct
  SITE 1: note = peptide may or may not be N-term modified
  SITE 6: note = peptide may or may not be C-term modified
AAERPG

SEQ ID NO: 186 - protein, 6 aa, synthetic construct
  SITE 1: note = peptide may or may not be N-term modified
  SITE 6: note = peptide may or may not be C-term modified
AERPGE

SEQ ID NO: 187 - protein, 6 aa, synthetic construct
  SITE 1: note = peptide may or may not be N-term modified
  SITE 6: note = peptide may or may not be C-term modified
ERPGEA

SEQ ID NO: 188 - protein, 6 aa, synthetic construct
  SITE 1: note = peptide may or may not be N-term modified
  SITE 6: note = peptide may or may not be C-term modified
RPGEAA

SEQ ID NO: 189 - protein, 6 aa, synthetic construct
  SITE 1: note = peptide may or may not be N-term modified
  SITE 6: note = peptide may or may not be C-term modified
PGEAAV

SEQ ID NO: 190 - protein, 6 aa, synthetic construct
  SITE 1: note = peptide may or may not be N-term modified
  SITE 6: note = peptide may or may not be C-term modified
GEAAVA

SEQ ID NO: 191 - protein, 5 aa, synthetic construct
  SITE 1: note = peptide may or may not be N-term modified
  SITE 5: note = peptide may or may not be C-term modified
GAQFS

SEQ ID NO: 192 - protein, 5 aa, synthetic construct
  SITE 1: note = peptide may or may not be N-term modified
  SITE 5: note = peptide may or may not be C-term modified
AQFSK

SEQ ID NO: 193 - protein, 5 aa, synthetic construct
  SITE 1: note = peptide may or may not be N-term modified
  SITE 5: note = peptide may or may not be C-term modified
QFSKT

SEQ ID NO: 194 - protein, 5 aa, synthetic construct
  SITE 1: note = peptide may or may not be N-term modified
  SITE 5: note = peptide may or may not be C-term modified
FSKTA

SEQ ID NO: 195 - protein, 5 aa, synthetic construct
  SITE 1: note = peptide may or may not be N-term modified
  SITE 5: note = peptide may or may not be C-term modified
SKTAA

SEQ ID NO: 196 - protein, 5 aa, synthetic construct
  SITE 1: note = peptide may or may not be N-term modified
  SITE 5: note = peptide may or may not be C-term modified
KTAAK

SEQ ID NO: 197 - protein, 5 aa, synthetic construct
  SITE 1: note = peptide may or may not be N-term modified
  SITE 5: note = peptide may or may not be C-term modified
TAAKG

SEQ ID NO: 198 - protein, 5 aa, synthetic construct
  SITE 1: note = peptide may or may not be N-term modified
  SITE 5: note = peptide may or may not be C-term modified
AAKGE

SEQ ID NO: 199 - protein, 5 aa, synthetic construct
  SITE 1: note = peptide may or may not be N-term modified
  SITE 5: note = peptide may or may not be C-term modified
AKGEA

SEQ ID NO: 200 - protein, 5 aa, synthetic construct
  SITE 1: note = peptide may or may not be N-term modified
  SITE 5: note = peptide may or may not be C-term modified
KGEAA

SEQ ID NO: 201 - protein, 5 aa, synthetic construct
  SITE 1: note = peptide may or may not be N-term modified
  SITE 5: note = peptide may or may not be C-term modified
GEAAA

SEQ ID NO: 202 - protein, 5 aa, synthetic construct
  SITE 1: note = peptide may or may not be N-term modified
  SITE 5: note = peptide may or may not be C-term modified
EAAAE

SEQ ID NO: 203 - protein, 5 aa, synthetic construct
  SITE 1: note = peptide may or may not be N-term modified
  SITE 5: note = peptide may or may not be C-term modified
AAAER

SEQ ID NO: 204 - protein, 5 aa, synthetic construct
  SITE 1: note = peptide may or may not be N-term modified
  SITE 5: note = peptide may or may not be C-term modified
AAERP

SEQ ID NO: 205 - protein, 5 aa, synthetic construct
  SITE 1: note = peptide may or may not be N-term modified
  SITE 5: note = peptide may or may not be C-term modified
AERPG

SEQ ID NO: 206 - protein, 5 aa, synthetic construct
  SITE 1: note = peptide may or may not be N-term modified
  SITE 5: note = peptide may or may not be C-term modified
ERPGE

SEQ ID NO: 207 - protein, 5 aa, synthetic construct
  SITE 1: note = peptide may or may not be N-term modified
  SITE 5: note = peptide may or may not be C-term modified
RPGEA

SEQ ID NO: 208 - protein, 5 aa, synthetic construct
  SITE 1: note = peptide may or may not be N-term modified
  SITE 5: note = peptide may or may not be C-term modified
PGEAA

SEQ ID NO: 209 - protein, 5 aa, synthetic construct
  SITE 1: note = peptide may or may not be N-term modified
  SITE 5: note = peptide may or may not be C-term modified
GEAAV

SEQ ID NO: 210 - protein, 5 aa, synthetic construct
  SITE 1: note = peptide may or may not be N-term modified
  SITE 5: note = peptide may or may not be C-term modified
EAAVA

SEQ ID NO: 211 - protein, 4 aa, synthetic construct
  SITE 1: note = peptide may or may not be N-term modified
  SITE 4: note = peptide may or may not be C-term modified
GAQF

SEQ ID NO: 212 - protein, 4 aa, synthetic construct
  SITE 1: note = peptide may or may not be N-term modified
  SITE 4: note = peptide may or may not be C-term modified
AQFS

SEQ ID NO: 213 - protein, 4 aa, synthetic construct
  SITE 1: note = peptide may or may not be N-term modified
  SITE 4: note = peptide may or may not be C-term modified
QFSK

SEQ ID NO: 214 - protein, 4 aa, synthetic construct
  SITE 1: note = peptide may or may not be N-term modified
  SITE 4: note = peptide may or may not be C-term modified
FSKT

SEQ ID NO: 215 - protein, 4 aa, synthetic construct
  SITE 1: note = peptide may or may not be N-term modified
  SITE 4: note = peptide may or may not be C-term modified
SKTA

SEQ ID NO: 216 - protein, 4 aa, synthetic construct
  SITE 1: note = peptide may or may not be N-term modified
  SITE 4: note = peptide may or may not be C-term modified
KTAA

SEQ ID NO: 217 - protein, 4 aa, synthetic construct
  SITE 1: note = peptide may or may not be N-term modified
  SITE 4: note = peptide may or may not be C-term modified
TAAK

SEQ ID NO: 218 - protein, 4 aa, synthetic construct
  SITE 1: note = peptide may or may not be N-term modified
  SITE 4: note = peptide may or may not be C-term modified
AAKG

SEQ ID NO: 219 - protein, 4 aa, synthetic construct
  SITE 1: note = peptide may or may not be N-term modified
  SITE 4: note = peptide may or may not be C-term modified
AKGE

SEQ ID NO: 220 - protein, 4 aa, synthetic construct
  SITE 1: note = peptide may or may not be N-term modified
  SITE 4: note = peptide may or may not be C-term modified
KGEA

SEQ ID NO: 221 - protein, 4 aa, synthetic construct
  SITE 1: note = peptide may or may not be N-term modified
  SITE 4: note = peptide may or may not be C-term modified
GEAA

SEQ ID NO: 222 - protein, 4 aa, synthetic construct
  SITE 1: note = peptide may or may not be N-term modified
  SITE 4: note = peptide may or may not be C-term modified
EAAA

SEQ ID NO: 223 - protein, 4 aa, synthetic construct
  SITE 1: note = peptide may or may not be N-term modified
  SITE 4: note = peptide may or may not be C-term modified
AAAE

SEQ ID NO: 224 - protein, 4 aa, synthetic construct
  SITE 1: note = peptide may or may not be N-term modified
  SITE 4: note = peptide may or may not be C-term modified
AAER

SEQ ID NO: 225 - protein, 4 aa, synthetic construct
  SITE 1: note = peptide may or may not be N-term modified
  SITE 4: note = peptide may or may not be C-term modified
AERP

SEQ ID NO: 226 - protein, 4 aa, synthetic construct
  SITE 1: note = peptide may or may not be N-term modified
  SITE 4: note = peptide may or may not be C-term modified
ERPG

SEQ ID NO: 227 - protein, 4 aa, synthetic construct
  SITE 1: note = peptide may or may not be N-term modified
  SITE 4: note = peptide may or may not be C-term modified
RPGE

SEQ ID NO: 228 - protein, 4 aa, synthetic construct
  SITE 1: note = peptide may or may not be N-term modified
  SITE 4: note = peptide may or may not be C-term modified
PGEA

SEQ ID NO: 229 - protein, 4 aa, synthetic construct
  SITE 1: note = peptide may or may not be N-term modified
  SITE 4: note = peptide may or may not be C-term modified
GEAA

SEQ ID NO: 230 - protein, 4 aa, synthetic construct
  SITE 1: note = peptide may or may not be N-term modified
  SITE 4: note = peptide may or may not be C-term modified
EAAV

SEQ ID NO: 231 - protein, 4 aa, synthetic construct
  SITE 1: note = peptide may or may not be N-term modified
  SITE 4: note = peptide may or may not be C-term modified
AAVA

SEQ ID NO: 232 - protein, 24 aa, synthetic construct
  SITE 1: note = peptide may or may not be N-term modified
  SITE 24: note = peptide may or may not be C-term modified
GTAPAAEGAGAEVKRASAEAKQAF

SEQ ID NO: 233 - protein, 12 aa, synthetic construct
  SITE 1: note = peptide may or may not be N-term modified
  SITE 12: note = peptide may or may not be C-term modified
GKQFSKTAAKGE

SEQ ID NO: 234 - protein, 12 aa, synthetic construct
  SITE 1: note = peptide may or may not be N-term modified
  SITE 12: note = peptide may or may not be C-term modified
GAQFSKTKAKGE

SEQ ID NO: 235 - protein, 12 aa, synthetic construct
  SITE 1: note = peptide may or may not be N-term modified
  SITE 12: note = peptide may or may not be C-term modified
GKQFSKTKAKGE

SEQ ID NO: 236 - protein, 10 aa, synthetic construct
  SITE 1: note = peptide may or may not be N-term modified
  SITE 10: note = peptide may or may not be C-term modified
GAQASKTAAK

SEQ ID NO: 237 - protein, 12 aa, synthetic construct
  SITE 1: note = peptide may or may not be N-term modified
  SITE 12: note = peptide may or may not be C-term modified
GAQASKTAAKGE

SEQ ID NO: 238 - protein, 12 aa, synthetic construct
  SITE 1: note = peptide may or may not be N-term modified
  SITE 12: note = peptide may or may not be C-term modified
GAEFSKTAAKGE

SEQ ID NO: 239 - protein, 12 aa, synthetic construct
  SITE 1: note = peptide may or may not be N-term modified
  SITE 12: note = peptide may or may not be C-term modified
GAQFSKTAAAGE

SEQ ID NO: 240 - protein, 12 aa, synthetic construct
  SITE 1: note = peptide may or may not be N-term modified
  SITE 12: note = peptide may or may not be C-term modified
GAQFSKTAAKAE

SEQ ID NO: 241 - protein, 12 aa, synthetic construct
  SITE 1: note = peptide may or may not be N-term modified
  SITE 12: note = peptide may or may not be C-term modified
GAQFSKTAAKGA

SEQ ID NO: 242 - protein, 10 aa, synthetic construct
  SITE 1: note = peptide may or may not be N-term modified
  SITE 10: note = peptide may or may not be C-term modified
AAQFSKTAAK

SEQ ID NO: 243 - protein, 10 aa, synthetic construct
  SITE 1: note = peptide may or may not be N-term modified
  SITE 10: note = peptide may or may not be C-term modified
GAAFSKTAAK

SEQ ID NO: 244 - protein, 10 aa, synthetic construct
  SITE 1: note = peptide may or may not be N-term modified
  SITE 10: note = peptide may or may not be C-term modified
GAQFAKTAAK

SEQ ID NO: 245 - protein, 10 aa, synthetic construct
  SITE 1: note = peptide may or may not be N-term modified
  SITE 10: note = peptide may or may not be C-term modified
GAQFSATAAK

SEQ ID NO: 246 - protein, 10 aa, synthetic construct
  SITE 1: note = peptide may or may not be N-term modified
  SITE 10: note = peptide may or may not be C-term modified
KAATKSFQAG

SEQ ID NO: 247 - protein, 10 aa, synthetic construct
  SITE 1: note = peptide may or may not be N-term modified
  SITE 10: note = peptide may or may not be C-term modified
GAQFSKAAAK

SEQ ID NO: 248 - protein, 10 aa, synthetic construct
  SITE 1: note = peptide may or may not be N-term modified
  SITE 10: note = peptide may or may not be C-term modified
GAQFSKTAAA

SEQ ID NO: 249 - protein, 10 aa, synthetic construct
  SITE 1: note = peptide may or may not be N-term modified
  SITE 10: note = peptide may or may not be C-term modified
GAQFSATAAA

SEQ ID NO: 250 - protein, 8 aa, synthetic construct
  SITE 1: note = peptide may or may not be N-term modified
  SITE 8: note = peptide may or may not be C-term modified
GAQASKTA

SEQ ID NO: 251 - protein, 4 aa, synthetic construct
  SITE 1: note = peptide may or may not be N-term modified
  SITE 4: note = peptide may or may not be C-term modified
AAGE

SEQ ID NO: 252 - protein, 10 aa, synthetic construct
  SITE 1: note = peptide may or may not be N-term modified
  SITE 10: note = peptide may or may not be C-term modified
GKASQFAKTA